(19) United States
(12) Patent Application Publication
(10) Pub. No.: US 2018/0313853 A1
(43) Pub. Date: Nov. 1, 2018

(54) CHIRAL CONVERSION OF AMYLOID PROTEINS ASSOCIATED WITH DISEASES

(71) Applicant: The Regents of the University of California, Oakland, CA (US)

(72) Inventor: Jevgenij A. Raskatov, Santa Cruz, CA (US)

(21) Appl. No.: 15/754,942

(22) PCT Filed: Aug. 26, 2016
(86) PCT No.: PCT/US2016/048993

§ 371 Date: Feb. 23, 2018

Related U.S. Application Data
(60) Provisional application No. 62/210,757, filed on Aug. 27, 2015.

(51) Int. Cl. G01N 33/68; A61K 38/17; A61P 25/28; C07K 14/47

(52) U.S. Cl. CPC G01N 33/6896; C07K 14/4711; A61P 25/28; A61K 38/1716

(57) ABSTRACT

Provided are pharmaceutical formulations that include an amyloid polypeptide including at least one D-amino acid, and a pharmaceutically acceptable carrier. Also provided are kits that include the pharmaceutical formulations. Therapeutic methods that employ the pharmaceutical compositions are also provided, as are methods of forming racemic amyloid polypeptide aggregates involving the contacting of all-L amyloid polypeptide aggregates (e.g., oligomers) with amyloid polypeptides that include at least one D-amino acid. Methods for reducing solubility of an all-L amyloid polypeptide in a fluid, methods for characterizing an amyloid polypeptide of interest, and methods for removing an amyloid polypeptide from a bodily fluid, are also provided.

### CROSS-REFERENCE TO RELATED APPLICATIONS

This application claims the benefit of U.S. Provisional Patent Application Ser. No. 62/210,757, filed Aug. 27, 2015, and U.S. Provisional Patent Application Ser. No. 62/365,841, filed Jul. 22, 2016, the disclosures of which are herein incorporated by reference in their entireties.

### BACKGROUND OF THE INVENTION

### Field of the Invention

The present invention relates to the field of protein chemistry, in particular to methods for modifying the activity or behavior of a protein by contacting it with peptides and other compounds with an opposite chirality, in whole or in part.

### Related Art

Presented below is background information on certain aspects of the present invention as they may relate to technical features referred to in the detailed description, but not necessarily described in detail. That is, individual compositions or methods used in the present invention may be described in greater detail in the publications and patents discussed below, which may provide further guidance to those skilled in the art for making or using certain aspects of the present invention as claimed. The discussion below should not be construed as an admission as to the relevance or the prior art effect of the patents or publications described.

Amyloid β (Aβ) Alzheimer's disease (AD) is a major neurodegenerative disorder that affects over 35 million people worldwide. Reflecting the increase in life expectancy, these numbers continue to rise, while no cure exists. Aβ is an aggregation-prone polypeptide of 36-49 amino acids in length, which has been strongly implicated in the mechanism of Alzheimer's disease, the second major culprit of the disease progression being hyperphosphorylation of the τ protein with resultant formation of insoluble neurofibrillary tangles. Aβ42 is widely regarded as the most toxic Aβ entity in Alzheimer's, which has been attributed to its high aggregation propensity. A hallmark of the disorder is the progressive deterioration of the patient's ability to establish long-term memories. This is rooted in the increasing damage to the brain hippocampal tissue, which is crucial for the consolidation of the memory trace. A recent seminal study showed that exposure of rat hippocampal slices to oligomers of all-L Aβ, but not the opposite enantiomer (all-D), Aβ42, impaired memory trace formation.

As discussed in Scheidt et al., “Solid-state NMR Reveals a Close Structural Relationship between Amyloid-β Protofibrils and Oligomers,” J Biol Chem. 2012 Jun. 29; 287(27): 22822-22826, Alzheimer's disease is characterized by extracellular deposition of plaques of Aβ peptides in the brain. These protein aggregates are composed of mature Aβ fibrils, which represent the end product of a long, complex, and not well understood fibrillation process. The fibrillation pathway initiates with soluble unstructured monomeric Aβ peptides, which are converted into oligomers, protofibrils, and finally into mature fibrils. Recently, interest in the transient Aβ intermediate structures has been growing rapidly because these species are considered to represent the cytotoxic intermediates in Alzheimer's disease.

As discussed below, in certain aspects, the present methods and materials include the use of D—
(a) β-Amyloid (Aβ);
(b) Type 2 diabetes (amylin) amyloid;
(c) Alpha-synuclein (SNCA) amyloid;
(d) TTR, and
(e) Huntingtin containing polyglutamine repeats, or,

amino acids in a synthetic amyloid polypeptide. Use of such a synthetic amyloid polypeptide can alter the formation of higher order structures comprising assembled natural L-amyloid polypeptide. In addition, the present materials can be used to isolate and characterize L-amyloid polypeptide.

Amylin (human islet amyloid polypeptide). Type 2 diabetes is a glucose metabolic disorder with over 300 million diseased patients worldwide. The two disease hallmarks are the insensitivity towards and the underproduction of the principal regulator of glucose metabolism, insulin. Type 2 diabetes is a multifactorial disease, in which aggregation of amylin appears as a strong causal factor. This represents another amyloid useful with the present methods and materials.

As discussed below, these diseases are exemplary, and the present methods may be applied to other amyloid-associated diseases.

### SPECIFIC PATENTS AND PUBLICATIONS

Ciccotosto et al., “Stereospecific interactions are necessary for Alzheimer disease amyloid-β toxicity,” Neurobiol Aging, 32:235-248 (2011) discloses that a D-handed enantiomer of Aβ42 (D-Aβ42) was synthesized, and its biophysical and neurotoxic properties were compared to the wild-type Aβ42 (L-Aβ42). Cell binding studies show both peptides bound to cultured cortical neurons. However, only L-Aβ42 was neurotoxic and inhibited long term potentiation indicating L-Aβ42 requires a stereospecific target to mediate toxicity. The lipid phosphatidylserine was identified as a potential target. Annexin V, which has very high affinity for externalized phosphatidylserine, significantly inhibited L-Aβ42 but not D-Aβ42 binding to the cultured cortical neurons and significantly rescued L-Aβ42 neurotoxicity. This suggests that Aβ mediated toxicity in Alzheimer disease is dependent upon Aβ binding to phosphatidylserine on neuronal cells.

Sawaya et al, “Atomic structures of amyloid cross-β spines reveal varied steric zippers,” Nature 447:453-457 (2007) discloses that amyloid fibrils formed from different proteins, each associated with a particular disease, contain a common cross-β spine. In particular, the fibril-forming segment GNNQQNY of the yeast prion protein Sup35, was recently revealed by X-ray microcrystallography. It is a pair of β-sheets, with the facing side chains of the two sheets interdigitated in a dry ‘steric zipper’. In this paper, the authors report some 30 other segments from fibril-forming proteins that form amyloid-like fibrils, microcrystals, or usually both. These include segments from the Alzheimer's amyloid-β and tau proteins, the PrP prion protein, insulin, islet amyloid polypeptide (IAPP), lysozyme, myoglobin, α-synuclein and β2-microglobulin, suggesting that common structural features are shared by amyloid diseases at the molecular level.

Kurouski et al., “Is Supramolecular Filament Chirality the Underlying Cause of Major Morphology Differences in Amyloid Fibrils?” J. Am. Chem. Soc. 136:2302-2312 (Jan. 9, 2014) discloses a study of the formation and development of amyloid fibrils in solution, using vibrational circular dichroism (VCD). Previously for insulin, it has been demonstrated that the sign of the VCD band pattern from filament chirality can be controlled by adjusting the pH of the incubating solution, above pH 2 for “normal” left-hand-helical filaments and below pH 2 for “reversed” right-hand-helical filaments. In this paper, VCD intensity has been observed for three additional amyloid fibrils, namely apo-α-lactalbumin, the HET-s (218-289) prion-forming domain, and TTR (105-115) peptide fragment from transthyretin. In a separate publication, similarly enhanced VCD has also been reported for a number of polyglutamine (polyQ) fibrils from Q15 to Q45. The result is strong evidence that the chiral supramolecular organization of filaments is the principal underlying cause of the morphological heterogeneity of amyloid fibrils.

### BRIEF SUMMARY OF THE INVENTION

The following brief summary is not intended to include all features and aspects of the present invention, nor does it imply that the invention must include all features and aspects discussed in this summary.

Provided are pharmaceutical formulations that include an amyloid polypeptide including at least one D-amino acid, and a pharmaceutically acceptable carrier. Also provided are kits that include the pharmaceutical formulations. Therapeutic methods that employ the pharmaceutical compositions are also provided, as are methods of forming racemic amyloid polypeptide aggregates involving the contacting of all-L amyloid polypeptide aggregates (e.g., all-L amyloid polypeptide oligomers) with amyloid polypeptides that include at least one D-amino acid. Methods for reducing solubility of an all-L amyloid polypeptide in a fluid, methods for characterizing an amyloid polypeptide of interest, and methods for removing an amyloid polypeptide from a bodily fluid, are also provided.

In certain aspects, the present disclosure provides a method for reducing solubility of an all L-amyloid polypeptide in a fluid, comprising: (a) contacting the all L-amyloid polypeptide in a fluid with a synthetic polypeptide having an amino acid sequence essentially identical to the all L-amyloid polypeptide, except that it contains D-amino acids, and (b) incubating the L-amyloid polypeptide with the synthetic polypeptide under conditions in which a complex of L-amyloid polypeptide with synthetic polypeptide is formed, whereby the complex has a solubility less than a complex formed of all L-amyloid polypeptides.

In certain embodiments, the method described above further includes the forming of a complex that contains at least one of 10%, 20%, 30%, 40%, or 50% synthetic polypeptide, the remainder being L-amyloid polypeptide; and/or the complex contains a synthetic polypeptide consisting essentially of all D-amino acids.

In other words, the synthetic polypeptide single chain non-covalently and spontaneously binds (under suitable conditions) with the target L-amyloid polypeptide and forms different higher order multimers. In certain embodiments, the method described above further includes a method where the L-amyloid polypeptide is a native amyloid polypeptide, and the fluid is blood (or a blood component) or cerebral spinal fluid. In certain embodiments, the methods described above further include a step of removing the complex from the fluid.

In certain embodiments, the methods described above further include methods where the fluid is from a host organism, and depleted fluid from the step of removing the complex is returned to the host. The host organism may be a human or other mammal.

In certain embodiments, the methods described above further includes a step of incubating the L-amyloid polypeptide with the synthetic polypeptide and further immobilizing the synthetic polypeptide on a plurality of beads and mixing the beads with the fluid. As will be understood from the present description, such a method will facilitate the isolation of the L-amyloid polypeptide and/or the complex containing it. In certain embodiments, the methods described above further include a step as defined above, wherein the beads are magnetic beads and are removed from the fluid by a magnetic material mixed with the fluid and removed from the fluid.

In certain embodiments, the methods described above further include the use of an amyloid polypeptide that is essentially identical to a polypeptide selected from the group consisting of: (a) β-Amyloid; (b) Type 2 diabetes (amylin) amyloid; (c) Alpha-synuclein (SNCA) amyloid; (d) TTR and (e) Huntingtin protein containing polyglutamine repeats, or, an amyloid polypeptide that has an identical primary amino acid sequence to the L-amyloid polypeptide.

As will be apparent from the following description, in certain aspects, the present disclosure provides the synthetic D-amino acid polypeptides having primary sequences substantially identical to those described above.

In certain embodiments, the present disclosure provides a method for characterizing an amyloid polypeptide of interest, comprising the steps of: (a) contacting the amyloid polypeptide of interest with a second amyloid polypeptide comprising D-amino acids and having an essentially identical sequence to the amyloid polypeptide of interest; (b) forming an aggregate between the amyloid polypeptide of interest with the second amyloid polypeptide; and (c) measuring the amount of aggregation that has formed. Such a method may further comprise a step wherein the measuring is done with a nuclear magnetic resonance (NMR) spectroscopic technique and wherein the amyloid polypeptide of interest and the second amyloid polypeptide comprising D-amino acids are chemically linked to different NMR labels. In certain embodiments, the inventive method described above further comprises a method as described above wherein the amyloid polypeptide of interest is selected from the group consisting of: (a) β-Amyloid; (b) Type 2 diabetes (amylin) amyloid; (c) Alpha-synuclein (SNCA) myloid; (d) TTR, and (e) Huntingtin containing polyglutamine repeats.

In certain embodiments, the inventive methods described herein further include a step of removing an amyloid polypeptide from a bodily fluid, comprising the step of contacting the bodily fluid with a synthetic polypeptide substantially identical in sequence to the amyloid polypeptide, but comprising at least a segment thereof of L-amino acids, wherein the synthetic polypeptide is immobilized to permit removal of the synthetic polypeptide in the form of a complex, the method further comprising forming the complex between the amyloid polypeptide in the synthetic polypeptide which permits removal of the complex from the bodily fluid. In this method, in certain embodiments, the synthetic polypeptide is immobilized on a bead.

The present disclosure further includes a synthetic amyloid polypeptide including a portion of L-amino acids and at least one portion of D-amino acids, where the portion of D-amino acids exhibits a higher affinity for a counterpart amyloid peptide than a corresponding portion of L-amino acids.

The present disclosure further includes the various synthetic amyloid polypeptides for use with any of the presently described methods. These synthetic amyloid polypeptides may include, in certain embodiments, a portion of L-amino acids and a contiguous stretch of 5-15 D-amino acids. These synthetic amyloid polypeptides may also be prepared as a pharmaceutical formulation for intravenous, intrathecal or intracranial administration.

In certain embodiments, the present disclosure provides a method for identifying an amyloid polypeptide binding compound, comprising: (a) providing a solution containing an amyloid polypeptide of interest; (b) adding to the mixture a second amyloid polypeptide having an essentially identical sequence to the amyloid polypeptide of interest, further having a defined portion of 1 to 10 D-amino acids and measuring aggregation formation; (c) repeating step (b) with a third amyloid polypeptide having an essentially identical sequence to the amyloid polypeptide of interest, further having a defined portion of 1 to 10 D-amino acids adjacent to the defined portion in step (b); and (d) comparing results of aggregation formation in steps (b) and (c).

### BRIEF DESCRIPTION OF THE DRAWINGS

FIG. 1A-1D is a schematic representation of pseudoracemate NMR experiments to investigate aggregation as a function of chirality. A distinction is drawn between pseudo-racemic (FIG. 1A) and pseudo-enantiopure (FIG. 1B) dimers. A range of tetrameric assemblies is conceivable (FIG. 1C). The exchange between the NMR-invisible fibrillary state (FIG. 1D) and the constituent monomer can be measured, applying the recently developed DEST NMR pulse sequence. Atomic force and scanning electron microscopy experiments, molecular modeling, dynamic light and small angle X-ray scattering approaches, and isothermal titration microcalorimetry measurements may be used to further support the analysis. As shown in FIG. 1C, one may prepare oligomers (tetramers) of D-L-racemic mixtures, or (FIG. 1D) protofibrils of racemic D-L mixtures, etc.

FIG. 2 is a schematic representation of the use of magnetic nanoparticles (MNP), decorated with varying densities of D-amino acid containing amyloid or a partial chiral mutant (either Aβ or amylin) 210. The thus immobilized polypeptides 210 offer nucleation sites, which will result in chemical affinity capture of solubilized native all L-amyloid 212. Magnetic dialysis approaches can then be employed to subsequently remove the nanoparticles and the precipitate. In addition to improved binding affinity, the use of D-amino acids should confer protease resistance upon the employed peptidic scaffolds.

FIG. 3 is a diagram that illustrates that there are two characterized modes of amyloid-associated toxicity, identified for Aβ and amylin, and exerted upon neurons and beta-cells, respectively. Left: Amyloid fibrils are typically straight and unbranched and are formed from an assembly of protofilaments 2-5 nm wide. X-ray diffraction analysis has indicated a characteristic structure, the β-cross motif, in which the polypeptide chains form β-strands oriented perpendicular to the long axis of the fibril, and β-sheets propagating in the fibril direction. Fenton-type chemistry refers to a metal-catalyzed redox reaction, which yields toxic reactive oxygen species, such as illustrated here with H₂O₂. Right: amyloid oligomers interact with lipid bilayers, disrupting membrane integrity and leading to deregulation of ion homeostasis. These two functional aspects of the amyloid manifolds can be studied as a function of chirality.

FIG. 4A, 4B is a schematic diagram of the creation of libraries of partial chiral mutants in order to optimize affinities of non-natural amyloids towards their cognate all-L counterparts (Aβ and amylin, respectively). This is termed for convenience a “chiral slider approach”. One introduces the chiral slider with a length of 6 amino acids in a sliding window of sequence within the peptide of interest. Other slider sizes and combinations thereof can also be used.

FIG. 5 is a schematic model of changes in Aβ transport across the blood-brain barrier, showing how all-L-Aβ fibrils found in a subject may be scavenged by all-D-Aβ proteins (polypeptides as described). The blood-brain barrier is represented by the center area 502, which divides the blood compartment (left column) and the brain compartment (right column). In the Aβ-expressing transgenic animal, all-L-Aβ (closed circles 504) is imported from the blood to the brain to a greater degree than it is exported from the brain to the blood. At 504 there is an equilibrium between L-Aβ in the blood and brain. When all-D-Aβ peptides (open circles 506) are administered intravenously, the balance of Aβ shifts to favor export of the all-L-Aβ from the brain to the blood. Also, intravenous administration of all-D-Aβ allows reduction of a blood concentration of L-Aβ. This is accomplished by tying up the free L-Aβ and complexing it with corresponding D-amino acid containing D-Aβ. Once the L-Aβ has been cleared, the law of mass action will tend to cause transport of all-D Aβ administered IV into the brain compartment, as shown at 508.

FIG. 6 is a schematic illustration of various D-L-amyloid mixtures being evaluated for transport across the blood brain barrier. The schematic shows a receptor of advanced glycation end-products (RAGE) mediates the Aβ transport across the blood brain barrier (BBB). Chiral substitutions in Aβ may impair this process. One may analyze the transport dependence on chiral substitution pattern (size and location) within Aβ to determine novel strategies to breach the blood brain barrier, and gain further mechanistic understanding for this important receptor.

FIG. 7 shows Thioflavin T (ThT) assay results indicating accelerated fibril formation for racemic Aβ as compared to all-L-Aβ and all-D-Aβ.

FIG. 8 provides transmission electron microscopy (TEM) images showing fibril morphology for all-L-Aβ, all-D-Aβ, and racemic Aβ.

FIG. 9 shows the results of a PICUP (Photo-Induced Cross-Linking of Unmodified Proteins) experiment indicating a shift toward formation of higher molecular weight aggregates in racemic Aβ as compared to the two individual enantiomers.

### DETAILED DESCRIPTION

### Overview

Disclosed herein, according to certain embodiments, is the preparation and use of D-isomers of amyloid peptides. The amyloid peptides of interest are expressed in vivo, and therefore are all L-isomers. Further, the amyloid peptides of interest naturally form, in vivo, complexes, also referred to as plaques. Still further, the formed racemic complexes of the peptide multimers or fibrils are thought to behave in a fundamentally different way in vivo. Their principal distinctions are accelerated fibril formation, enhanced stability and reduced activity, which are thought to result in the attenuation of toxicity.

Aggregation-prone (amyloidogenic) proteins sample a range of assemblies of varying sizes. These can be broadly classified as low-weight oligomers, high-weight oligomers and fibrils. Together they form ensembles of states (i.e., manifolds) that exist in dynamic equilibrium. In contrast to the commonly pursued anti-aggregation approaches to disease-associated amyloid plaques, the invention described herein builds on the recent findings that oligomeric assemblies of disease-causing amyloids are the principal toxic species, and that the associated large (fibrillary) amyloid aggregates represent protective reservoirs. Using the present invention, the equilibrium of structural components may be shifted away from oligomers by stabilizing the fibrillary state, thereby attenuating the toxicity of oligomeric amyloid assemblies. As an added benefit, this approach can provide novel mechanistic tools that may be applied to investigating a wide range of amyloid-associated disorders, including Parkinson's disease and Huntington's disease.

According to the present methods, toxicity of aggregation-prone polypeptides, such as amyloid β in Alzheimer's disease and amylin in type 2 diabetes, can be reduced by enhancement of amyloid aggregation. Employing the respective opposing enantiomers of the amyloids to de-populate the oligomeric fractions of their manifolds underlies such strategies. This can be expected from the present disclosure because it is known that equimolar mixtures of the two enantiomers of a chiral substance (a racemate) possess higher stabilities and reduced reactivities, compared to their enantiomerically pure counterparts. The chiral inactivation hypothesis is derived from fundamentals of crystallography, physical organic, organometallic and polymer chemistry, and holds the potential of yielding novel therapeutic strategies. In particular, the racemic mixtures of proteins, small molecules, amyloids or inorganic salts exhibit properties that make them more stable and, hence, less reactive than their enantiomerically pure counterparts. Oligomeric species represent the principal toxicity carrier, and the extended state may, in fact, serve a protective scavenging function. This phenomenon appears to be independent of the nature of the amyloid-associated disorder and the cognate amyloidogenic polypeptide.

The present invention comprises the introduction of the opposite (non-native) enantiomer of a toxic, aggregation-prone polypeptide which will enhance the aggregation of the amyloidogenic polypeptide, thus reducing the population of the associated toxicity-carrying oligomeric states. The methods disclosed herein exploit properties of chiral conversion (from L- to D-) to neutralize the toxic oligomeric assemblies within amyloid manifolds. It should be noted that this approach is diametrically opposed to the existing small molecule-based therapeutic strategies, which commonly focus on attenuating aggregation.

The present therapeutic strategies can be applied to diverse (age-related) pathologies, including Huntington's disease (the Huntingtin protein), Parkinson's disease (α-synuclein), Alzheimer's disease (amyloid β), type 2 diabetes (amylin), and cataracts (γ-crystallin). A characteristic feature of an amyloidogenic polypeptide is the existence of a manifold of aggregates with varying size and composition. Distinctions are frequently made between low weight oligomers, high weight oligomers, protofibrils and plaques. Recent investigations strongly suggest that oligomers are causative to disease, while plaques may possess protective effects, exerted by scavenging the oligomeric species. This is possibly the root of the failure of plaque-solubilizing drug candidates in recent clinical trials that focused on Alzheimer's disease.

### Definitions

Unless defined otherwise, all technical and scientific terms used herein have the same meaning as commonly understood by those of ordinary skill in the art to which this invention belongs. Although any methods and materials similar or equivalent to those described herein can be used in the practice or testing of the present invention, the preferred methods and materials are described. Generally, nomenclatures utilized in connection with, and techniques of, cell and molecular biology and chemistry are those well-known and commonly used in the art. Certain experimental techniques, not specifically defined, are generally performed according to conventional methods well known in the art and as described in various general and more specific references that are cited and discussed throughout the present specification. For purposes of clarity, the following terms are defined below.

Ranges: For conciseness, any range set forth is intended to include any sub-range within the stated range, unless otherwise stated. As a non-limiting example, a range of 120 to 250 is intended to include a range of 120-121, 120-130, 200-225, 121-250 etc. The term “about” has its ordinary meaning of approximately and may be determined in context by experimental variability. In case of doubt, the term “about” means plus or minus 5% of a stated numerical value. Similarly, a stated range, for example, of 90 to 95 present should be read as a possible range of 91-92, 90-92, 90-93, etc.

The term “aggregate” refers to a complex formed by a natural propensity of amyloid polypeptides to self-assemble. The aggregates, in Alzheimer's disease (AD), form plaques that predominately consist of the aggregates. It is believed that Aβ oligomerization occurs via distinct intermediates, including oligomers of 3-50 Aβ monomers, annular oligomers, protofibrils, fibrils and plaques.

The term “amyloid polypeptide” refers to a protein or protein fragment that is expressed or processed in a cell to a form in which multiple polypeptides may form into amorphous aggregates, fibrils, and oligomers. The term “amyloid” is used herein in its accepted definition, e.g., they may be heterogeneous within a specific protein precursor. Amyloid is a generic term referring to a group of diverse but specific intra- and extracellular protein deposits which are associated with a number of different diseases. Though diverse in their occurrence, all amyloid deposits have common morphologic properties, including that they stain with specific dyes (e.g., Congo red), and have a characteristic birefringent appearance (sometimes characterized as “red-green”) in polarized light after staining. They also share common ultrastructural features and common x-ray diffraction and infrared spectra.

Amyloidosis can be classified clinically as primary, secondary, familial and/or isolated. Isolated forms of amyloidosis are those that tend to involve a single organ system. Different amyloids are also characterized by the type of protein present in the deposit. For example, neurodegenerative diseases such as scrapie, bovine spongiform encephalitis, Creutzfeldt-Jakob disease and the like are characterized by the appearance and accumulation of a protease-resistant form of a prion protein (referred to as AScr or PrP-27) in the central nervous system. Similarly, Alzheimer's disease, another neurodegenerative disorder, is characterized by congophilic angiopathy, neuritic plaques and neurofibrillary tangles, all of which have the characteristics of amyloids. In this case, the plaque and blood vessel amyloid is formed by the amyloid beta protein. Other diseases, such as juvenile and adult-onset diabetes, complications of long-term hemodialysis and sequelae of long-standing inflammation or plasma cell dyscrasias are characterized by the accumulation of amyloids systemically. In each of these cases, a different amyloidogenic protein is involved in amyloid deposition.

The term “amyloid polypeptide” includes, unless the context is to the contrary, one of the following:

1. Alzheimer's' Amyloid

For example, Aβ peptides are heterogeneous short hydrophobic peptides (<5 kDa) ranging between 36 and 49 amino acids in length. Several different Aβ peptide species exist, but Aβ₁₋₄₀ (Aβ₄₀) is the most abundant. Aβ₄₀ peptides contain 16 amino acid residues in the amino extracellular domain of APP, and 24 amino acids in the membrane-spanning domain. Aβ₄₂ has two additional hydrophobic residues, Ile and Ala, and accounts for only 2-5% of Aβ peptides. Aβ₄₂ peptides are more hydrophobic and fibrillogenic, have a higher aggregation potential and are the principal species deposited in the brain.

As used herein, “Amyloid β polypeptide” or “Aβ polypeptide” denotes polypeptides of 36 to 49 amino acids (Aβ36, Aβ37, Aβ38, Aβ39, Aβ40, Aβ41, Aβ42, Aβ43, Aβ44, Aβ45, Aβ46, Aβ47, Aβ48, and Aβ49) that are the main component of the amyloid plaques found in the brains of Alzheimer's disease (AD) patients. The peptides result from the amyloid precursor protein (APP), which is cleaved by beta secretase and gamma secretase to yield Aβ. The amino acid sequence of the wild-type Aβ49 peptide is:

(SEQ ID NO: 1)
DAEFRHDSGYEVHHQKLVFFAEDVGSNKGAIIGLMVGGVVIATVIVITL

The other wild-type Aβ peptides are shorter versions of the Aβ49 peptide. For example, the wild-type Aβ42 peptide has the following amino acid sequence:

(SEQ ID NO: 2)
DAEFRHDSGYEVHHQKLVFFAEDVGSNKGAIIGLMVGGVVIA

Also by way of example, the amino acid sequence of the wild-type A1340 peptide is:

(SEQ ID NO: 3)
DAEFRHDSGYEVHHQKLVFFAEDVGSNKGAIIGLMVGGVV

As used herein, the terms “beta-amyloid protein,” “amyloid beta protein,” “Aβ,” “Aβ polypeptide,” “Aβ peptide” and “Aβ protein” all refer to a protein produced by neurons and glial cells in the brain. Aβ is found in the brain plaques of patients with Alzheimer's disease, head trauma and Down's syndrome, and is also found normally in the spinal fluid and blood (See, e.g., Haass and Selkoe, Cell 75:1039 [1993]; Teller et al., Nature Med., 2:93 [1996]).

2. Type 2 Diabetes (Amylin) Amyloid

Amylin, which is a 37-amino acid long polypeptide, is co-secreted with insulin by the pancreatic β-cells during normal cell function. Amylin has been demonstrated to induce β-cell death in cell culture and result in development of diabetes in transgenic mice that homozygously express the human polypeptide. Mechanistic parallels have been noted between type 2 diabetes and Alzheimer's disease, which extend beyond the fact that both are aging-related protein folding disorders. Specifically, the oligomeric pre-fibrillar state of amylin has been characterized as the chief carrier of insult to the pancreatic beta-cells, the principal target of amylin toxicity. As with Alzheimer's Aβ, amylin fibrils serve a protective reservoir function. Interestingly, a positive correlation between the propensities towards development of Alzheimer's disease and type 2 diabetes has been reported, implying that the two manifolds are capable of cross-potentiating.

Amylin (AIPP) has the following amino acid sequence:

(SEQ ID NO: 4, UniProtKB- P10997 (IAPP_HUMAN))
MGILKLQVFL IVLSVALNHL KATPIESHQV EKRKCNTATC
ATQRLANFLV HSSNNFGAIL SSTNVGSNTY GKRNAVEVLK
REPLNYLPL

3. Alpha-Synuclein (SNCA) Amyloid

This protein is thought to be involved in the regulation of dopamine release and transport. It induces the fibrillization of the microtubule-associated protein tau, and reduces neuronal responsiveness to various apoptotic stimuli, leading to decreased caspase-3 activation.

It has the following amino acid sequence:

(SEQ ID NO: 5)
MDVFMKGLSK AKEGVVAAAE KTKQGVAEAA GKTKEGVLYV
GSKTKEGVVH GVATVAEKTK EQVTNVGGAV VTGVTAVAQK
TVEGAGSIAA ATGFVKKDQL GKNEEGAPQE GILEDMPVDP
DNEAYEMPSE EGYQDYEPEA.

It is associated with Parkinson's disease type 4. This is a complex neurodegenerative disorder with manifestations ranging from typical Parkinson's disease to dementia with Lewy bodies. Clinical features include Parkinsonian symptoms (resting tremor, rigidity, postural instability and bradykinesia), dementia, diffuse Lewy body pathology, autonomic dysfunction, hallucinations and paranoia. It is also associated with Dementia Lewy body (DLB1). This disease is caused by mutations affecting the gene represented above. It is a neurodegenerative disorder characterized by mental impairment leading to dementia, parkinsonism, fluctuating cognitive function, visual hallucinations, falls, syncopal episodes, and sensitivity to neuroleptic medication. Brainstem or cortical intraneuronal accumulations of aggregated proteins (Lewy bodies) are the only essential pathologic features. Patients may also have hippocampal and neocortical senile plaques, sometimes in sufficient number to fulfill the diagnostic criteria for Alzheimer's disease.

4. Huntingtin Protein (Involved in Huntington's Disease), Isoform CRA a, has the Following Amino Acid Sequence:

(SEQ ID NO: 6)
MATLEKLMKAFESLKSFQQQQQQQQQQQQQQQQQQQQQQQPPPPPPPP
PPPQLPQPPPQAQPLLPQPQPPPPPPPPPPGPAVAEEPLHRPKKELSA
TKKDRVNHCLTICENIVAQSVRNSPEFQKLLGIAMELFLLCSDDAESD
VRMVADECLNKVIKALMDSNLPRLQLELYKEIKKNGAPRSLRAALWRF
AELAHLVRPQKCRPYLVNLLPCLTRTSKRPEESVQETLAAAVPKIMAS
FGNFANDNEIKVLLKAFIANLKSSSPTIRRTAAGSAVSICQHSRRTQY
FYSWLLNVLLGLLVPVEDEHSTLLILGVLLTLRYLVPLLQQQVKDTSL
KGSFGVTRKEMEVSPSAEQLVQVYELTLHHTQHQDHNVVTGALELLQQ
LFRTPPPELLQTLTAVGGIGQLTAAKEESGGRSRSGSIVELIAGGGSS
CSPVLSRKQKGKVLLGEEEALEDDSESRSDVSSSALTASVKDEISGEL
AASSGVSTPGSAGHDIITEQPRSQHTLQADSVDLASCDLTSSATDGDE
EDILSHSSSQVSAVPSDPAMDLNDGTQASSPISDSSQTTTEGPDSAVT
PSDSSEIVLDGTDNQYLGLQIGQPQDEDEEATGILPDEASEAFRNSSM
ALQQAHLLKNMSHCRQPSDSSVDKFVLRDEATEPGDQENKPCRIKGDI
GQSTDDDSAPLVHCVRLLSASFLLTGGKNVLVPDRDVRVSVKALALSC
VGAAVALHPESFFSKLYKVPLDTTEYPEEQYVSDILNYIDHGDPQVRG
ATAILCGTLICSILSRSRFHVGDWMGTIRTLTGNTFSLADCIPLLRKT
LKDESSVTCKLACTAVRNCVMSLCSSSYSELGLQLIIDVLTLRNSSYW
LVRTELLETLAEIDFRLVSFLEAKAENLHRGAHHYTGLLKLQERVLNN
VVIHLLGDEDPRVRHVAAASLIRLVPKLFYKCDQGQADPVVAVARDQS
SVYLKLLMHETQPPSHFSVSTITRIYRGYNLLPSITDVTMENNLSRVI
AAVSHELITSTTRALTFGCCEALCLLSTAFPVCIWSLGWHCGVPPLSA
SDESRKSCTVGMATMILTLLSSAWFPLDLSAHQDALILAGNLLAASAP
KSLRSSWASEEEANPAATKQEEVWPALGDRALVPMVEQLFSHLLKVIN
ICAHVLDDVAPGPAIKAALPSLTNPPSLSPIRRKGKEKEPGEQASVPL
SPKKGSEASAASRQSDTSGPVTTSKSSSLGSFYHLPSYLKLHDVLKAT
HANYKVTLDLQNSTEKFGGFLRSALDVLSQILELATLQDIGKCVEEIL
GYLKSCFSREPMMATVCVQQLLKTLFGTNLASQFDGLSSNPSKSQGRA
QRLGSSSVRPGLYHYCFMAPYTHFTQALADASLRNMVQAEQENDTSGW
FDVLQKVSTQLKTNLTSVTKNRADKNAIHNHIRLFEPLVIKALKQYTT
TTCVQLQKQVLDLLAQLVQLRVNYCLLDSDQVFIGFVLKQFEYIEVGQ
FRESEAIIPNIFFFLVLLSYERYHSKQIIGIPKIIQLCDGIMASGRKA
VTHAIPALQPIVHDLFVLRGTNKADAGKELETQKEVVVSMLLRLIQYH
QVLEMFILVLQQCHKENEDKWKRLSRQIADIILPMLAKQQMHIDSHEA
LGVLNTLFEILAPSSLRPVDMLLRSMFVTPNTMASVSTVQLWISGILA
ILRVLISQSTEDIVLSRIQELSFSPYLISCTVINRLRDGDSTSTLEEH
SEGKQIKNLPEETFSRFLLQLVGILLEDIVTKQLKVEMSEQQHTFYCQ
ELGTLLMCLIHIFKSGMFRRITAAATRLFRSDGCGGSFYTLDSLNLRA
RSMITTHPALVLLWCQILLLVNHTDYRWWAEVQQTPKRHSLSSFTKLL
SPQMSGEEEDSDLAAKLGMCNREIVRRGALILFCDYVCQNLHDSEHLT
WLIVNHIQDLISLSHEPPVQDFISAVHRNSAASGLFIQAIQSRCENLS
TPTMLKKTLQCLEGIHLSQSGAVLTLYVDRLLCTPFRVLARMVDILAC
RRVEMLLAANLQSSMAQLPMEELNRIQEYLQSSGLAQRHQRLYSLLDR
FRLSTMQDSLSPSPPVSSHPLDGDGHVSLETVSPDKDWYVHLVKSQCW
TRSDSALLEGAELVNRIPAEDMNAFMMNSEFNLSLLAPCLSLGMSEIS
GGQKSALFEAAREVTLARVSGTVQQLPAVHHVFQPELPAEPAAYWSKL
NDLFGDAALYQSLPTLARALAQYLVVVSKLPSHLHLPPEKEKDIVKFV
VATLEALSWHLIHEQIPLSLDLQAGLDCCCLALQLPGLWSVVSSTEFV
THACSLIYCVHFILEAVAVQPGEQLLSPERRTNTPKAISEEEEEVDPN
TQNPKYITAACEMVAEMVESLQSVLALGHKRNSGVPAFLTPLLRNIII
SLARLPLVNSYTRVPPLVWKLGWSPKPGGDFGTAFPEIPVEFLQEKEV
FKEFIYRINTLGWTSRTQFEETWATLLGVLVTQPLVMEQEESPPEEDT
ERTQINVLAVQAITSLVLSAMTVPVAGNPAVSCLEQQPRNKPLKALDT
RFGRKLSIIRGIVEQEIQAMVSKRENIATHHLYQAWDPVPSLSPATTG
ALISHEKLLLQINPERELGSMSYKLGQVSIHSVWLGNSITPLREEEWD
EEEEEEADAPAPSSPPTSPVNSRKHRAGVDIHSCSQFLLELYSRWILP
SSSARRTPAILISEVVRSLLVVSDLFTERNQFELMYVTLTELRRVHPS
EDEILAQYLVPATCKAAAVLGMDKAVAEPVSRLLESTLRSSHLPSRVG
ALHGVLYVLECDLLDDTAKQLIPVISDYLLSNLKGIAHCVNIHSQQHV
LVMCATAFYLIENYPLDVGPEFSASIIQMCGVMLSGSEESTPSIIYHC
ALRGLERLLLSEQLSRLDAESLVKLSVDRVNVHSPHRAMAALGLMLTC
MYTGKEKVSPGRTSDPNPAAPDSESVIVAMERVSVLFDRIRKGFPCEA
RVVARILPQFLDDFFPPQDIMNKVIGEFLSNQQPYPQFMATVVYKVFQ
TLHSTGQSSMVRDWVMLSLSNFTQRAPVAMATWSLSCFFVSASTSPWV
AAILPHVISRMGKLEQVDVNLFCLVATDFYRHQIEEELDRRAFQSVLE
VVAAPGSPYHRLLTCLRNVHKVTTC

Huntington's disease (HD) is caused by a mutated form of the Huntingtin gene, where excessive (more than 36) CAG repeats result in formation of an unstable protein. Walker F O (January 2007). “Huntington's disease”. Lancet 369 (9557): 218-28. doi:10.1016/S0140-6736(07)60111-1.

These expanded repeats lead to production of a Huntingtin protein that contains an abnormally long polyglutamine tract at the N-terminus. The underlined sequence above is for the reader's convenience and comparison with the sequence below, illustrating a sequence having the glutamine repeats. A normal N terminus is underlined above and indicated below, with the abnormal repeats as follows:

(SEQ ID NO: 7)
MATLEKLMKAFESLKSFQQQQQQQQQQQQQQQQQQQQQQQPPPPPPPP
PPPQLPQPPPQAQPLLPQPQPPPPPPPPPPGPAVAEEPLHRPKKELSA
TKKDRVNHCLTICENIVAQSVRNSPEFQKLLGIAMELFLLCSDDAESD
VRMVADECLNKVIKALMDSNLPRLQLELYKEIKKNGAPRSLRAALWRF
AELAHLVRPQKCRPYLVNLLPCLTRT

The above is as set forth completely in GenBank Accession No. NM_002111

Huntington's disease falls in a class of neurodegenerative disorders known as trinucleotide repeat disorders or polyglutamine disorders. The key sequence which is found in Huntington's disease is a trinucleotide repeat expansion of glutamine residues beginning at the 18th amino acid. In unaffected individuals, this contains between 9 and 35 glutamine residues with no adverse effects. (Macdonald M (1993). “A novel gene containing a trinucleotide repeat that is expanded and unstable on Huntington's disease chromosomes. The Huntington's Disease Collaborative Research Group”. Cell 72 (6): 971-83) However, 36 or more residues produce an erroneous form of Htt, mHtt (standing for mutant Htt). Reduced penetrance is found in counts 36-39 (Chong S S, Almqvist E, Telenius H, LaTray L, Nichol K, Bourdelat-Parks B, Goldberg Y P, Haddad B R, Richards F, Sillence D, Greenberg C R, Ives E, Van den Engh G, Hughes M R, Hayden M R (February 1997). “Contribution of DNA sequence and CAG size to mutation frequencies of intermediate alleles for Huntington disease: evidence from single sperm analyses”. Human Molecular Genetics 6 (2): 301-9.)

5. TTR Transthyretin

Another amyloid polypeptide useful in the formulations and methods of the present disclosure is TTR transthyretin [Homo sapiens (human)], gene ID 7278, and its various polypeptide variants. An example is Chain A, Human Transthyretin (Prealbumin),

(SEQ ID NO: 8)
GPTGTGESKC PLMVKVLDAV RGSPAINVAV HVFRKAADDT
WEPFASGKTS ESGELHGLT EEEFVEGIYK VEIDTKSYWK
ALGISPFHEH AEVVFTANDS GPRRYTIAAL LSPYSYSTTA
VVTNPKE.

The three dimensional structure may be found at PDB: 1BMZ_A. It is further discussed at Peterson et al., “Inhibiting transthyretin conformational changes that lead to amyloid fibril formation.,” Proc Natl Acad Sci USA. 1998 Oct. 27; 95(22):12956-60. D-amino acid containing polypeptides essentially identical to the above can be used to stabilize or destabilize the conformation of this protein.

By way of further description, the Table below lists proteins that have amyloid characteristics, wherein the present methods and formulations may be used to modulate the interactions of the individual proteins, as described in detail below.

| | | Official |
|---|---|---|
| Disease | Protein Featured | abbreviation |
| Alzheimer's disease | Beta amyloid | Aβ |
| Diabetes mellitus type 2 | IAPP (Amylin) | AIAPP |
| Parkinson's disease | Alpha-synuclein | none |
| Transmissible spongiform | PrPSc | APrP |
| encephalopathy e.g. Bovine | | |
| spongiform encephalopathy | | |
| Fatal Familial Insomnia | PrPSc | APrP |
| Huntington's Disease | Huntingtin | none |
| Medullary carcinoma of the | Calcitonin | ACal |
| thyroid | | |
| Cardiac arrhythmias, Isolated | Atrial natriuretic factor | AANF |
| atrial amyloidosis | | |
| Atherosclerosis | Apolipoprotein AI | AApoA1 |
| Rheumatoid arthritis | Serum amyloid A | AA |
| Aortic medial amyloid | Medin | AMed |
| Prolactinomas | Prolactin | APro |
| Familial amyloid | Transthyretin | ATTR |
| polyneuropathy | | |
| Hereditary non-neuropathic | Lysozyme | ALys |
| systemic amyloidosis | | |
| Dialysis related amyloidosis | Beta-2 microglobulin | Aβ2M |
| Finnish amyloidosis | Gelsolin | AGel |
| Lattice corneal dystrophy | Keratoepithelin | AKer |
| Cerebral amyloid angiopathy | Beta amyloid | Aβ |
| Cerebral amyloid angiopathy | Cystatin | ACys |
| (Icelandic type) | | |
| systemic AL amyloidosis | Immunoglobulin light chain | AL |
| | AL | |
| Sporadic Inclusion Body | S-IBM | none |
| Myositis | | |

The term “amyloid polypeptide aggregate” is used herein to refer to any one of a number of possible higher order structures between separate amyloid polypeptide chains. For example, Alzheimer's disease is characterized by extracellular deposition of plaques of amyloid-β (Aβ) polypeptides in the brain. These protein aggregates are composed of mature Aβ fibrils, which represent the end product of a long, complex, and not well understood fibrillation process. The fibrillation pathway initiates with soluble unstructured monomeric Aβ peptides, which are converted into oligomers, protofibrils, and finally into mature fibrils. Recently, interest in the transient Aβ intermediate structures has been growing rapidly because these species are considered to represent the cytotoxic intermediates in Alzheimer's disease (taken from Scheidt, et al. (2012) “Solid-state NMR supports the model of intramolecular hydrogen bonds in Aβ protofibrils”, J. Biol. Chem. 287(27): 22822-22826.

The term “bodily fluid” includes all fluids obtained from a mammalian body, including, for example, blood, plasma, urine, lymph, gastric juices, bile, serum, saliva, sweat, and spinal and brain fluids. Furthermore, the bodily fluids may be either processed (e.g., serum) or unprocessed.

The terms “essentially identical” or “essential identity” as used herein denote a characteristic of a polypeptide sequence, wherein the polypeptide comprises a sequence that has at least 60 percent sequence identity, at least 85 percent identity, at least 90 percent identity, at least 95 percent identity, or at least 99 percent sequence identity as compared to a reference sequence over a comparison window of the entire peptide length. Essential identity further involves a conservative substitution of an amino acid. “Essentially all” in reference to a polypeptide sequence means at least 80%, the range, as discussed below, including at least 90%, 99% and 100%.

The term “native amyloid polypeptide” is an all L-amyloid. It also refers to an amyloid polypeptide as described above and that is created in vivo by cellular activity, e.g. a cellular cleavage process (e.g. γ secretase acting in a cellular environment). It may be distinguished from, and is distinct from, in vitro synthesis, e.g. FMOC chemistry). A native amyloid polypeptide may have a variety of in vivo-formed polypeptides, i.e. different mutations or cleavage products. Unless the context indicates otherwise, the term refers to a single chain of a polypeptide, even though it may be in aggregated form.

The term “polypeptide” is used herein in its conventional sense, i.e., a polymer in which the monomers are amino acids and are joined together through amide bonds, alternatively referred to as a polypeptide. When the amino acids are α-amino acids, either the L-optical isomer or the D-optical isomer are used as specified in the present application. Additionally, unnatural amino acids, for example, β-alanine, phenylglycine and homoarginine are also meant to be included. Standard abbreviations for amino acids are used (as described below). The polypeptides specifically described here are human, unless otherwise indicated. It will be appreciated that the present methods may be adapted to other species susceptible to amyloid diseases.

The term “synthetic polypeptide” refers to a synthetic polypeptide that may include non-natural amino acids and other modifications such as substituting one or more L-amino acids for a corresponding D-amino acid.

In view of the discussions here, it can be understood that the term “chirality” can refer to an individual amino acid, e.g. an L-alanine or a D-alanine. Accordingly, a single polypeptide can have chirality based on the characteristic of the amino acids in the chain; i.e. a beta amyloid (Aβ) synthesized as a synthetic polypeptide of all D-amino acids will have a chirality on the polypeptide level. Finally, amyloids can assemble as supramolecular structures such as tetramers, oligomers, and fibrils. An Aβ fibril has been referred to as having chirality based on the helix structure of the amino acids. The helices may be left-handed or right-handed.

Accordingly, as used herein, chirality can refer to these higher order structures, but in all cases will comprise D-amino acids. As described below, amyloid proteins exhibit a self-assembling property, and the use of D-amino acids in amyloid polypeptide chains will convert a naturally occurring chain, either in a single chain form or in a higher order form, into a racemic mixture of L- and D-containing both optical isomers.

### General Methods

Described below are methods for studying, characterizing, and ultimately reducing the toxicity of amyloid polypeptides in a host organism. As is known, amyloid polypeptides spontaneously form aggregates between different polypeptide chains. In this invention, a native amyloid polypeptide is mixed with a D-amino acid containing amyloid polypeptide of the same species, i.e. amyloid β associated with Alzheimer's disease, or amyloid secreted by pancreatic beta cells. The D-containing synthetic polypeptide may be 100% identical in sequence to the native amyloid polypeptide. In other cases, it will not be 100% identical to the native amyloid polypeptide but will be essentially identical in sequence, along the length of the polypeptide.

For purposes of clarity, the term essentially identical is defined above. Certain guidelines may be used in making the changes to the present sequences.

General Description of Sequence Similarities

It is contemplated that the present methods will be carried out with amyloid peptides that are 100% D-enantiomers, and are 100% identical in amino acid sequence to the native amyloid peptide. However, it is further contemplated that for a variety of reasons, the present invention may be carried out with variations in D-content and primary amino acid sequence. The present methods using D-enantiomers are described as using D-enantiomers that are “essentially identical” to their corresponding naturally occurring amyloid peptide. For example, the term “essentially identical” in the context of a naturally occurring human beta-amyloid includes an identity based on the size of the peptide. For human beta-amyloid 1-42 (H14A), namely, D A E F R H D S G Y E V H A Q K L V F F A E D V G S N K G A I I G L M V G G V V I A, (SEQID NO: 9) essentially identical refers to the H14A mutation, or 97.6% (41/42) identity. Similarly, the term may refer to the Iowa mutation, D23N (See, Van Nostrand, W. et al. J. Biol. Chem. 276, 32860 (2001). Other mutations in a naturally occurring beta-amyloid are included, e.g. D7H, E22Q (Dutch mutation).

For purposes of a synthetic D-beta amyloid used to induce aggregation and reduce solubility, the synthetic peptide may be a fragment of the native beta amyloid polypeptide being treated, as small as 90% in size identity to the native amyloid polypeptide.

Conservative amino acid substitutions are those that take place within a family of amino acids that are related in their side chains. Genetically encoded amino acids are generally divided into families: (1) acidic=aspartate, glutamate; (2) basic=lysine, arginine, and histidine; (3) non-polar=alanine, valine, leucine, isoleucine, proline, phenylalanine, methionine, tryptophan; and (4) uncharged polar=glycine, asparagine, glutamine, cysteine, serine, threonine, and tyrosine. More preferred families are: serine and threonine are an aliphatic-hydroxy family; asparagine and glutamine are an amide-containing family; alanine, valine, leucine and isoleucine are an aliphatic family; phenylalanine, tryptophan, and tyrosine are an aromatic family, and cysteine and methionine are a sulfur-containing side chain family. For example, it is reasonable to expect that an isolated replacement of a leucine with an isoleucine or valine, an aspartate with a glutamate, a threonine with a serine, or a similar replacement of an amino acid with a structurally related amino acid will not have a major effect on the binding or properties of the resulting molecule, especially if the replacement does not involve an amino acid within a framework site. Preferred conservative amino acid substitution groups are: valine-leucine-isoleucine, phenylalanine-tyrosine, lysine-arginine, alanine-valine, glutamic acid-aspartic acid, cysteine-methionine, and asparagine-glutamine.

D-amyloid beta peptide is commercially available, e.g. from Sigma-Aldrich, or may be synthesized by known methods. See, e.g. Anderson U.S. Pat. No. 7,655,602, “Peptides comprising aromatic D-amino acids and methods of use,” for methods of preparing the peptides described here.

The standard single letter and three letter codes for amino acids are used herein and are as follows:

| | | |
|---|---|---|
| A (Ala) Alanine | C (Cys) Cysteine | D (Asp) Aspartic acid |
| E (Glu) Glutamic acid | F (Phe) Phenylalanine | G (Gly) Glycine |
| H (His) Histidine | I (Ile) Isoleucine | K (Lys) Lysine |
| L (Leu) Leucine | M (Met) Methionine | N (Asn) Asparagine |
| P (Pro) Proline | Q (Gln) Glutamine | R (Arg) Arginine |
| S (Ser) Serine | T (Thr) Threonine | V (Val) Valine |
| W (Trp) Tryptophan | Y (Tyr) Tyrosine | |

As described above, the indicated residues may be the naturally occurring L amino acid, or a modification thereof, that is, a chemical modification, an optical isomer, or a link to a modifying group. It is contemplated that specific modifications may be made within the peptide that maintain the ability of the present peptides to specifically induce aggregation and reduce solubility of the native amyloid polypeptide being treated.

It is also contemplated that specific modifications may be made in a particular sequence in order to confer some additional desirable property to the peptide. Certain amino acids may be substituted for other amino acids in a protein structure without appreciable loss of peptide activity. Since it is the interactive capacity and nature of a peptide that defines that peptide's biological functional activity, certain amino acid sequence substitutions can be made even in a short peptide sequence and nevertheless obtain a peptide with like properties. It is thus contemplated by the inventor that various changes may be made in the sequence of the present synthetic peptides without appreciable loss of biological utility or activity and perhaps may enhance desired activities.

### PHARMACEUTICAL FORMULATIONS

According to certain embodiments, the present disclosure provides pharmaceutical formulations that include an amyloid polypeptide comprising at least one D-amino acid, and a pharmaceutically acceptable carrier.

In certain aspects, the amyloid polypeptide including at least one D-amino acid is a β-Amyloid (Aβ) polypeptide, a Type 2 diabetes (amylin) amyloid polypeptide, an Alpha-synuclein (SNCA) amyloid polypeptide, a transthyretin (TTR) polypeptide, a Huntingtin polypeptide, or a fragment thereof. According to certain embodiments, the amyloid polypeptide including at least one D-amino acid includes 70% or greater, 75% or greater, 80% or greater, 85% or greater, 90% or greater, 95% or greater, 99% or greater, or 100% amino acid sequence identity to a wild-type β-Amyloid polypeptide, a wild-type Type 2 diabetes (amylin) amyloid polypeptide, a wild-type Alpha-synuclein (SNCA) amyloid polypeptide, a wild-type transthyretin (TTR) polypeptide, a wild-type Huntingtin polypeptide, or a fragment thereof. In certain aspects, by “a fragment thereof” is meant a fragment of from 5 to 10 amino acids in length, 10 to 15 amino acids in length, 15 to 20 amino acids in length, 20 to 25 amino acids in length, 25 to 30 amino acids in length, 30 to 35 amino acids in length, 35 to 40 amino acids in length, 40 to 45 amino acids in length, 45 to 50 amino acids in length, or 50 or more amino acids in length.

According to certain embodiments, the amyloid polypeptide including at least one D-amino acid is an Aβ polypeptide of from 36 to 49 amino acids in length. By way of example, the Aβ polypeptide including at least one D-amino acid may be an Aβ42 polypeptide. Also by way of example, the Aβ polypeptide including at least one D-amino acid may be an Aβ40 polypeptide.

The amyloid polypeptides including at least one D-amino acid may include one or any desired number of D-amino acids. In certain aspects, the amyloid polypeptides including at least one D-amino acid include 2 or more D-amino acids. According to certain embodiments, the amyloid polypeptide including at least one D-amino acid includes 5% or more, 10% or more, 15% or more, 20% or more, 25% or more, 30% or more, 35% or more, 40% or more, 45% or more, 50% or more, 55% or more, 60% or more, 65% or more, 70% or more, 75% or more, 80% or more, 85% or more, 90% or more, 95% or more, 99% or more, or 100% D-amino acids across the entire length of the amyloid polypeptide.

The pharmaceutical formulations of the present disclosure generally include a therapeutically effective amount of the amyloid polypeptide including at least one D-amino acid. By “therapeutically effective amount” is meant a dosage sufficient to produce a desired result, e.g., an amount sufficient to effect beneficial or desired therapeutic (including preventative) results, such as a reduction in a symptom of a disease or disorder associated with all-L amyloid polypeptide aggregates (e.g., oligomers), as compared to a control. An effective amount can be administered in one or more administrations.

The amyloid polypeptide including at least one D-amino acid can be incorporated into a variety of formulations for therapeutic administration. More particularly, the conjugate can be formulated into pharmaceutical compositions by combination with appropriate, pharmaceutically acceptable carriers (e.g., excipients or diluents), and may be formulated into preparations in solid, semi-solid, liquid or gaseous forms, such as tablets, capsules, powders, granules, ointments, solutions, injections, inhalants and aerosols.

Pharmaceutical formulations that include the amyloid polypeptide including at least one D-amino acid may be prepared by mixing the amyloid polypeptide including at least one D-amino acid having the desired degree of purity with optional physiologically acceptable carriers, excipients, stabilizers, surfactants, buffers and/or tonicity agents. Acceptable carriers, excipients and/or stabilizers are nontoxic to recipients at the dosages and concentrations employed, and include buffers such as phosphate, citrate, and other organic acids; antioxidants including ascorbic acid, glutathione, cysteine, methionine and citric acid; preservatives (such as ethanol, benzyl alcohol, phenol, m-cresol, p-chlor-m-cresol, methyl or propyl parabens, benzalkonium chloride, or combinations thereof); amino acids such as arginine, glycine, ornithine, lysine, histidine, glutamic acid, aspartic acid, isoleucine, leucine, alanine, phenylalanine, tyrosine, tryptophan, methionine, serine, proline and combinations thereof; monosaccharides, disaccharides and other carbohydrates; low molecular weight (less than about 10 residues) polypeptides; proteins, such as gelatin or serum albumin; chelating agents such as EDTA; sugars such as trehalose, sucrose, lactose, glucose, mannose, maltose, galactose, fructose, sorbose, raffinose, glucosamine, N-methylglucosamine, galactosamine, and neuraminic acid; and/or non-ionic surfactants such as Tween, Brij Pluronics, Triton-X, or polyethylene glycol (PEG).

According to certain embodiments, the amyloid polypeptide including at least one D-amino acid is formulated for parenteral (e.g., intravenous, intrathecal, intracerebral, intra-cranial, intra-arterial, intraosseous, intramuscular, intracerebroventricular, subcutaneous, etc.) administration. In certain aspects, the conjugate is formulated for injection by dissolving, suspending or emulsifying the conjugate in an aqueous or non-aqueous solvent, such as vegetable or other similar oils, synthetic aliphatic acid glycerides, esters of higher aliphatic acids or propylene glycol; and if desired, with conventional additives such as solubilizers, isotonic agents, suspending agents, emulsifying agents, stabilizers and preservatives.

The pharmaceutical formulations of the present disclosure may be in a liquid form, a lyophilized form or a liquid form reconstituted from a lyophilized form, wherein the lyophilized preparation is to be reconstituted with a sterile solution prior to administration. The standard procedure for reconstituting a lyophilized composition is to add back a volume of pure water (typically equivalent to the volume removed during lyophilization); however solutions including antibacterial agents may be used for the production of pharmaceutical formulations for parenteral administration.

### Methods of Making Amyloid Polypeptides

Methods of making the amyloid polypeptides of the present disclosure are also provided. In certain aspects, the polypeptide is produced using a chemical synthesis technique. Where a polypeptide is chemically synthesized, the synthesis may proceed via liquid-phase or solid-phase. Solid phase polypeptide synthesis (SPPS), in which the C-terminal amino acid of the sequence may be attached to an insoluble support followed by sequential addition of the remaining amino acids in the sequence (including the relevant one or more D-amino acid(s)), is an example of a suitable method for the chemical synthesis of a polypeptide of the present disclosure. Various forms of SPPS, such as Fmoc and Boc, are available for synthesizing the polypeptide. For example, small insoluble, porous beads may be treated with functional units on which peptide chains are built. After repeated cycling of coupling/deprotection, the free N-terminal amine of a solid-phase attached is coupled to a single N-protected amino acid unit. This unit is then deprotected, revealing a new N-terminal amine to which a further amino acid may be attached. The polypeptide remains immobilized on the solid-phase and may undergo a filtration process before being cleaved off.

Once synthesized, the polypeptide can be purified according to standard procedures, including ammonium sulfate precipitation, affinity columns, column chromatography, high performance liquid chromatography (HPLC) purification, gel electrophoresis, and the like.

### Kits

As summarized above, the present disclosure provides kits. According to certain embodiments, the kits include the pharmaceutical formulations of the present disclosure, e.g., any pharmaceutical formulation described elsewhere herein (e.g., including any of the amyloid polypeptides including at least one D-amino acid described elsewhere herein). The kits find use, e.g., in practicing the methods of the present disclosure. For example, kits for practicing the subject methods may include a quantity of the pharmaceutical formulations of the present disclosure, present in unit dosages, e.g., ampoules, or a multi-dosage format. As such, in certain embodiments, the kits may include one or more (e.g., two or more) unit dosages (e.g., ampoules) of a pharmaceutical formulation that includes an amyloid polypeptide including at least one D-amino acid of the present disclosure. The term “unit dosage”, as used herein, refers to physically discrete units suitable as unitary dosages for human and animal subjects, each unit containing a predetermined quantity of the composition calculated in an amount sufficient to produce the desired effect. The amount of the unit dosage depends on various factors, such as the particular amyloid polypeptide employed, the effect to be achieved, and the pharmacodynamics associated with the amyloid polypeptide in the subject. In yet other embodiments, the kits may include a single multi dosage amount of the formulation.

Components of the kits may be present in separate containers, or multiple components may be present in a single container. A suitable container includes a single tube (e.g., vial), one or more wells of a plate (e.g., a 96-well plate, a 384-well plate, etc.), or the like.

According to certain embodiments, a kit of the present disclosure includes instructions for using the pharmaceutical formulation to treat an individual in need thereof. The instructions may be recorded on a suitable recording medium. For example, the instructions may be printed on a substrate, such as paper or plastic, etc. As such, the instructions may be present in the kits as a package insert, in the labeling of the container of the kit or components thereof (i.e., associated with the packaging or sub-packaging) etc. In other embodiments, the instructions are present as an electronic storage data file present on a suitable computer readable storage medium, e.g., portable flash drive, DVD, CD-ROM, diskette, etc. In yet other embodiments, the actual instructions are not present in the kit, but means for obtaining the instructions from a remote source, e.g. via the internet, are provided. An example of this embodiment is a kit that includes a web address where the instructions can be viewed and/or from which the instructions can be downloaded. As with the instructions, the means for obtaining the instructions is recorded on a suitable substrate.

### Therapeutic Methods

Also provided are methods that include administering to an individual in need thereof a therapeutically effective amount of a pharmaceutical formulation of the present disclosure, e.g., any pharmaceutical formulation described elsewhere herein (e.g., including any of the amyloid polypeptides including at least one D-amino acid described elsewhere herein).

In certain aspects, the methods of the present disclosure include administering the pharmaceutical formulation to an individual having a disease or disorder related to (e.g., caused by) all-L amyloid polypeptide aggregation (e.g., all-L amyloid polypeptide oligomers (e.g., all-L amyloid polypeptide intermediates of 100 kDa or less)), e.g., to treat such a disease or disorder. According to certain embodiments, the administering is effective to convert all-L amyloid polypeptide oligomers in the individual to higher molecular weight racemic amyloid polypeptide aggregates (that is, aggregates having a higher molecular weight than the all-L amyloid polypeptide oligomers prior to the administration). In certain aspects, the individual in need thereof has Alzheimer's Disease (AD), and the amyloid polypeptide that includes at least one D-amino acid included in the pharmaceutical formulation is an Aβ polypeptide of from 36 to 49 amino acids in length. For example, the Aβ polypeptide that includes at least one D-amino acid may be an Aβ42 polypeptide, an Aβ40 polypeptide, and/or the like.

By “therapeutically effective amount” is meant a dosage sufficient to produce a desired result, e.g., an amount sufficient to effect beneficial or desired therapeutic (including preventative) results, such as a reduction in a symptom of a disease or disorder associated with all-L amyloid polypeptide aggregates (e.g., cytotoxic all-L amyloid oligomers), as compared to a control. An effective amount can be administered in one or more administrations.

The pharmaceutical formulations of the present disclosure are administered to the individual using any available method and route suitable for drug delivery, including in vivo and ex vivo methods, as well as systemic and localized routes of administration. Conventional and pharmaceutically acceptable routes of administration include intranasal, intramuscular, intra-tracheal, subcutaneous, intradermal, topical application, ocular, intravenous, intra-arterial, nasal, oral, and other enteral and parenteral routes of administration. Routes of administration may be combined, if desired, or adjusted depending upon the amyloid polypeptide and/or the desired effect. The pharmaceutical formulation may be administered in a single dose or in multiple doses. In some embodiments, the pharmaceutical formulation is administered intrathecally. In some embodiments, the conjugate is administered intracranially. In some embodiments, the conjugate is administered intravenously. In some embodiments, the pharmaceutical formulation is administered orally. In some embodiments, the pharmaceutical formulation is administered via an inhalational route. In some embodiments, the pharmaceutical formulation is administered intranasally. In some embodiments, the pharmaceutical formulation is administered locally. In some embodiments, the pharmaceutical formulation is administered by injection, e.g., for systemic delivery (e.g., intravenous infusion) or to a local site.

Strategies for delivery of biomolecules via intrathecal administration are described in Miller et al. (2013) Lancet Neurol. 12(5):435-42. Macrophage-based delivery approaches such as those described in Dou et al. (2009) J. Immunol. 183(1):661-9, may also be employed. Focused ultrasound to reversibly open the blood-brain barrier for drug delivery, as described in Treat et al. (2007) Int. J. Cancer 121:901-7, may also be employed. Glycosylation may be used to facilitate delivery of the amyloid polypeptides including at least one D-amino acid to the brain. Such a delivery strategy is described in Deane et al. (2003) Nat. Med. 9(7):907-13.

A variety of individuals are treatable according to the subject methods. Generally such individuals are “mammals” or “mammalian,” where these terms are used broadly to describe organisms which are within the class mammalia, including the orders carnivore (e.g., dogs and cats), rodentia (e.g., mice, guinea pigs, and rats), and primates (e.g., humans, chimpanzees, and monkeys). In some embodiments, the individual is a human.

By “treat” or “treatment” is meant at least an amelioration of the symptoms associated with the pathological condition afflicting the individual, where amelioration is used in a broad sense to refer to at least a reduction in the magnitude of a parameter, e.g., symptom, associated with the pathological condition being treated, such as disease or disorder associated with (e.g., caused by) all-L amyloid polypeptide aggregation (e.g., cytotoxic all-L amyloid polypeptide oligomers), where, e.g., the formation of higher molecular weight racemic amyloid polypeptide aggregates in the individual is beneficial. As such, treatment also includes situations where the pathological condition, or at least symptoms associated therewith, are completely inhibited, e.g., prevented from happening, or stopped, e.g. terminated, such that the individual no longer suffers from the pathological condition, or at least the symptoms that characterize the pathological condition.

Dosing is dependent on severity and responsiveness of the disease state to be treated. Optimal dosing schedules can be calculated from measurements of accumulation of the amyloid polypeptide including at least one D-amino acid in the body of the individual. The administering physician can determine optimum dosages, dosing methodologies and repetition rates. Optimum dosages may vary depending on the relative potency of the amyloid polypeptide including at least one D-amino acid, and can generally be estimated based on EC50s found to be effective in in vitro and in vivo animal models, etc. In general, dosage is from 0.01 μg to 100 g per kg of body weight, and may be given once or more daily, weekly, monthly or yearly. The treating physician can estimate repetition rates for dosing based on measured residence times and concentrations of the drug in bodily fluids or tissues. Following successful treatment, it may be desirable to have the subject undergo maintenance therapy to prevent the recurrence of the disease state, where the pharmaceutical formulation is administered in maintenance doses, once or more daily, to once every several months, once every six months, once every year, or at any other suitable frequency.

The therapeutic methods of the present disclosure may include administering a single type of amyloid polypeptide including at least one D-amino acid to an individual, or may include administering two or more types of amyloid polypeptides that include at least one D-amino acid to an individual (e.g., a cocktail of different amyloid polypeptides that include at least one D-amino acid). In certain aspects, an amyloid polypeptide that includes at least one D-amino acid is administered to the individual in combination with a second therapeutic agent, e.g., a second agent that modulates amyloid peptide aggregation in a desired manner. Such administration may include administering the amyloid polypeptide that includes at least one D-amino acid and the second agent concurrently (e.g., in the same formulation or different formulations), or administering the amyloid polypeptide and the second agent sequentially.

### Methods of Forming Racemic Amyloid Polypeptide Aggregates

As summarized above, methods of forming racemic amyloid polypeptide aggregates are also provided. According to certain embodiments, such methods include contacting aggregates (e.g., oligomers, such as all-L amyloid polypeptide intermediates of 100 kDa or less) including all-L amyloid polypeptides (e.g., all-L Aβ, such as all-L Aβ42) with amyloid polypeptides including at least one D-amino acid, where the amyloid polypeptides including at least one D-amino acid correspond to the all-L amyloid polypeptides, to form racemic amyloid polypeptide aggregates. In certain aspects, the racemic amyloid polypeptide aggregates have a higher molecular weight (e.g., a higher average molecular weight) than the aggregates (e.g., oligomers) including all-L amyloid polypeptides, prior to the contacting. According to such embodiments, the methods may be characterized as methods of forming racemic amyloid polypeptide aggregates having a higher molecular weight (e.g., a higher average molecular weight) than the starting aggregates (e.g., oligomers).

By “correspond to” is meant that the amyloid polypeptides including at least one D-amino acid are the same type of amyloid polypeptides as the all-L amyloid polypeptides present in the pre-contacted aggregates. For example, when the aggregates (e.g., oligomers) are aggregates of all-L polypeptides, the aggregates are contacted with Aβ polypeptides that include at least one D-amino acid. The amyloid polypeptides including at least one D-amino acid may have the same or different lengths as compared to the all-L amyloid polypeptides of the aggregates. By way of example, aggregates (e.g., oligomers) of all-L Aβ42 polypeptides may be contacted with Aβ42 polypeptides that include at least one D-amino acid, or alternatively or additionally, may be contacted with Aβ polypeptides that include at least one D-amino acid which have fewer (e.g., Aβ40) or more amino acids as compared to Aβ42.

In certain aspects, the contacting includes combining the aggregates (e.g., oligomers) including all-L amyloid polypeptides and the amyloid polypeptides including at least one D-amino acid under aggregation conditions in a container. Suitable aggregation conditions include, but are not limited to, the aggregation conditions described in detail in the Examples section herein below (e.g., Example 8 herein). Suitable containers include, but are not limited to, a single tube (e.g., vial), one or more wells of a plate (e.g., a 96-well plate, a 384-well plate, etc.), or the like.

In other aspects, the contacting occurs in vivo. For example, the contacting may include administering the amyloid polypeptides including at least one D-amino acid to an individual including the aggregates (e.g., oligomers) including all-L amyloid polypeptides. Any suitable route of administration may be employed. For example, the amyloid polypeptides including at least one D-amino acid may be administered via parenteral administration, e.g., intrathecal, intracranial, intravenous, or other suitable form of parenteral administration. Strategies for delivery of biomolecules via intrathecal administration are described in Miller et al. (2013) Lancet Neurol. 12(5):435-42. Macrophage-based delivery approaches such as those described in Dou et al. (2009) J. Immunol. 183(1):661-9, may also be employed. Focused ultrasound to reversibly open the blood-brain barrier for drug delivery, as described in Treat et al. (2007) Int. J. Cancer 121:901-7, may also be employed. Glycosylation may be used to facilitate delivery of the amyloid polypeptides including at least one D-amino acid to the brain. Such a delivery strategy is described in Deane et al. (2003) Nat. Med. 9(7):907-13.

In the present methods of forming racemic amyloid polypeptide aggregates, the amyloid polypeptides including at least one D-amino acid may include any of the features described hereinabove in the section relating to the pharmaceutical formulations of the present disclosure. For example, the amyloid polypeptides including at least one D-amino acid may be β-Amyloid (Aβ) polypeptide, Type 2 diabetes (amylin) amyloid polypeptides, Alpha-synuclein (SNCA) amyloid polypeptides, transthyretin (TTR) polypeptides, Huntingtin polypeptides, fragments thereof, or any combination thereof.

In certain aspects, the amyloid polypeptides including at least one D-amino acid include 2 or more D-amino acids. According to certain embodiments, the amyloid polypeptides including at least one D-amino acid include 5% or more, 10% or more, 15% or more, 20% or more, 25% or more, 30% or more, 35% or more, 40% or more, 45% or more, 50% or more, 55% or more, 60% or more, 65% or more, 70% or more, 75% or more, 80% or more, 85% or more, 90% or more, 95% or more, 99% or more, or 100% D-amino acids across the entire length of the amyloid polypeptide.

According to certain embodiments of the present methods of forming racemic amyloid polypeptide aggregates, the aggregates (e.g., oligomers) including all-L amyloid polypeptides include all-L β-Amyloid (Aβ) polypeptides (e.g., the aggregates may be cytotoxic oligomers of Aβ, such as Aβ42 cytotoxic oligomers), and the amyloid polypeptides including at least one D-amino acid are Aβ polypeptides including at least one D-amino acid. In certain aspects, the Aβ polypeptides including at least one D-amino acid are Aβ polypeptides of from 36 to 49 amino acids in length, e.g., Aβ42, Aβ40, and/or the like.

### EXAMPLES

### Example 1: Pseudoracemates for Altering Intra-Chain Arrangements of Amyloid Polypeptides

In order to select a particular D-peptide for contacting an L-amyloid polypeptide, one performs structural studies to investigate particle size, distribution, exchange kinetics and enthalpies of mixing in amyloid manifolds as a function of chirality. A pseudoracemate or a solid solution forms when the two enantiomers coexist in an unordered manner in the crystal. The term is also used to refer to a synthetically created racemic mixture. A racemate is optically inactive, meaning that there is no net rotation of plane-polarized light. Although the two enantiomers rotate plane-polarized light in opposite directions, the rotations cancel because they are present in equal amounts.

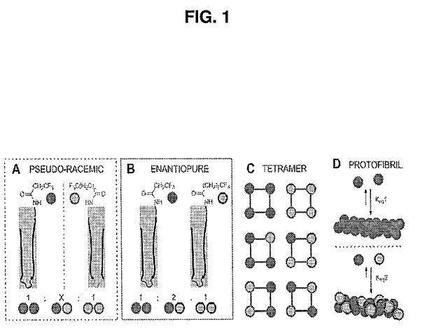

Nuclear magnetic resonance (NMR) represents a powerful tool to investigate molecular aggregation. The pseudoracemate approach shown in FIG. 1A-1D allows distinguishing the two enantiomers spectroscopically using different labels of alkyl fluorine indicated by solid circles and open circles. According to the present methods, different combinations of D- and L-amyloids can be created and studied as tetramers, and protofibrils. Protofibrils are intermediate structures between oligomers and fibrils, present in the growth phrase of a fibril. Amyloid fibrils are typically straight and unbranched and are formed from an assembly of protofilaments 2-5 nm wide. X-ray diffraction analysis has indicated a characteristic structure, the β-cross motif, in which the polypeptide chains form β-strands oriented perpendicular to the long axis of the fibril, and β-sheets propagating in the fibril direction. For further identification of protofibrils, see Dubnovitsky et al., “Amyloid-β Protofibrils: Size, Morphology and Synaptotoxicity of an Engineered Mimic,” PLoS ONE 8(10): 10.1371.

Pseudoracemates can be created by integrating enantiomer-characteristic isotope substitution patterns or performing minor derivatization, such as the integration of NMR-active F-19 nuclei (FIG. 1A-1D). The recently developed dark-state NMR approach (Fawzi, “Dark-state exchange saturation transfer,” Nature Methods 8, 997 (2011)) is used to analyze exchange kinetics with amyloid fibrils, which are invisible by NMR (FIG. 1D). For further study, one may also employ scanning electron microscopy to gain structural insight at the level of individual molecules. Molecular modeling can be employed as a tool to support experimentally determined structures. Dynamic light scattering, mass spectrometry and small angle X-ray scattering techniques may be applied to determine size distribution of aggregates that constitute the amyloid manifolds as a function of chirality. One may also conduct isothermal titration microcalorimetry measurements to determine thermodynamic parameters for the formation of racemic mixtures from enantiopure amyloid polypeptides.

### Example 2: Chemical Affinity Capture of Amyloid Polypeptides and Magnetic Dialysis

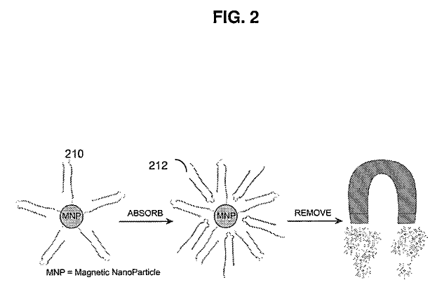

Referring now to FIG. 2, both amylin and Aβ are medicinally relevant targets, found in blood or other accessible fluid. The present invention includes the use of magnetic nanoparticles to remove amyloid from a fluid that may be present in or removed from a subject. The details of such a method in which the nanoparticles are decorated with an antibody is described in Wankhede et al., “Magnetic nanoparticles: an emerging technology for malignant brain tumor imaging and therapy,” Expert Rev. Clin. Pharmacol. 5(2) 173-186 (2012), which should be consulted for detail on the preparation of such particles and their delivery to either the blood or, by direct convection-enhanced delivery, to the brain. The magnetic nanoparticle is attached to a number of D-amyloids; these amyloids bind to L-amyloids in the fluid in which the particles are mixed. Then, external magnets are used to remove the complexes with both the original D-amyloids and the absorbed L-amyloids, which will bind to the D-amyloids immobilized on the nanoparticles.

The beads may utilize small particles of transition metals such as iron, nickel, copper, cobalt and manganese to form metal oxides which can be caused to have inducible magnetic properties in the presence of magnets which are transitory; such particles are termed paramagnetic or superparamagnetic. To form paramagnetic or superparamagnetic beads, metal oxides have been coated with polymers which are relatively stable in water. For example, U.S. Pat. No. 4,554,088 (Whitehead, et al.) discloses paramagnetic particles comprising a metal oxide core surrounded by a coat of polymeric silane; U.S. Pat. No. 5,356,713 (Charmot), discloses a magnetizable microsphere comprised of a core of magnetizable particles surrounded by a shell of a hydrophobic vinylaromatic monomer; and U.S. Pat. No. 5,395,688 (Wang) discloses a polymer core which has been coated with a mixed paramagnetic metal oxide-polymer layer. The disclosure of each is incorporated herein by reference. Another method utilizes a polymer core to adsorb metal oxide such as, for example, in U.S. Pat. No. 4,774,265 (Ugelstad), incorporated herein by reference. (See “Composite magnetic beads,” U.S. Pat. No. 5,834,121 for details and for details of removal steps. One may also use commercially available Dynabeads® magnetic separation technology, available from Thermofisher Scientific Inc.

The development of chemical affinity capture strategies to remove these polypeptides from the bloodstream would possess enormous therapeutic potential. Whereas amylin is co-secreted with insulin by pancreatic β-cells and released into the bloodstream, the biodistribution mechanism of Aβ is substantially more complex, since it is produced in the brain and has to cross the blood-brain barrier in order to enter the blood stream. Elaborate shuttling mechanisms exist, which enable the Aβ polypeptide to breach the blood-brain barrier and equilibrate between the two compartments. Specifically, the receptor for advanced glycation end-products, as well as the lipoprotein receptor proteins 1 and 2, are all membrane-bound proteins, which are integrated in the blood-brain barrier. The receptor for advanced glycation end-products is responsible for binding blood-borne Aβ and importing it into the brain, whereas the lipoprotein receptor proteins bind the brain-located Aβ polypeptide and export it back into the bloodstream. A dynamically equilibrating flow system is therewith established. Further details of the mechanism may be found in Zlokovic et al., Nat Rev Neurosci. 12:723-738 (2011).

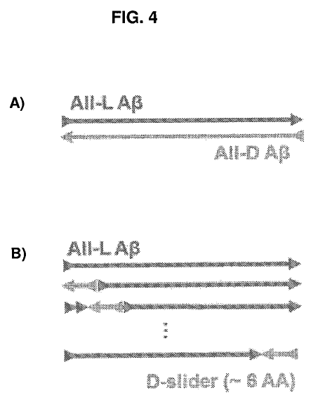

In the present example, one synthesizes nanoparticles decorated with all-D Aβ or the amylin polypeptide, or the corresponding partial chiral mutants (FIGS. 1A-1D and FIG. 4A, 4B). Size and seeding densities are optimized with regard to capturing their all-L counterparts.

Initial experiments are conducted in cell-free systems, and are then gradually expanded to defined cell mixtures and serum samples. This approach can serve to remove blood-borne amyloids from patients via magnetic dialysis. Magnetic dialysis is a recently developed technology that was shown to be highly effective at removing pathogens from blood, employing antibody-decorated magnetic nanoparticles. The present approach possesses two distinct advantages over the use of proteins: 1) Aβ and amylin are both relatively short polypeptides (below 50 amino acids), and can therefore be prepared readily, using solid phase synthesis protocols; and 2) as the frameworks are derived from D-amino acids, they will be resistant to blood-borne proteases, which is not the case for natural all-L frameworks.

### Example 3: Characterization of D- and Racemic Mixtures of Amyloid Polypeptides and Measurement of Membrane Disruption

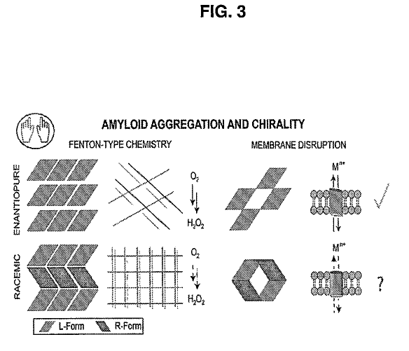

Here, one evaluates the properties and activities of an enantio-pure amyloid complex (protofibrils, all-L) compared with a corresponding mixture of D- and L-forms of the same aggregation. An amount of racemic mixture is evaluated for its ability to form higher order protofibrils (FIG. 3, left panel) or insert into lipid membranes (FIG. 3, right panel).

Both Aβ and amylin polypeptides are potent metal binders with sub-femtomolar affinity for copper, binding of which confers upon them the ability to engage in Fenton-type redox chemistry and produce harmful reactive oxygen species from molecular dioxygen. Aβ and amylin have been correlated to synapto- and β-cell-toxicity in Alzheimer's disease, and type 2 diabetes, respectively. As shown in FIG. 3, left panel, a Fenton-type redox reaction can be carried out by adding Cu⁺² (or Fe⁺³), producing, as shown, reactive species such as O₂ and H₂O₂.

The first reduction product of oxygen is the superoxide radical (O₂—). Carrying an unpaired electron, superoxide is a potent oxidizing agent. It has been found to react with numerous cellular structures, such as iron-sulfur clusters, unsaturated lipids, and nucleic acids. Superoxide can also cause biological damage by acting as a reductant, as in the Fenton reaction described below.

Reduction of superoxide yields hydrogen peroxide (H₂O₂). Hydrogen peroxide is also a powerful oxidant and is commonly used in first aid as a biocide to cleanse wounds, or in high concentrations as a bleaching agent. While superoxide carries a charge—and is thus unable to freely cross biological membranes—H₂O₂ is uncharged, and can diffuse across membranes as easily as water. Hydrogen peroxide is also more stable than superoxide, and can diffuse through a cell or tissue, causing damage at a distance from its point of origin.

In the presence of catalytic amounts of iron, superoxide and hydrogen peroxide can react to produce hydroxyl radical (OH.). Hydroxyl radical is one of the most potent oxidizing agents known. It is too reactive to diffuse far in a cellular environment rich in targets for oxidation. It is thought to cause damage in the vicinity of iron centers or other sites containing bound iron. Oxidation by hydroxyl radical results in the concomitant reduction of the radical to water.

The amyloids share two types of well-characterized aggregation-related activity. Referring now to FIG. 3 right panel, oligomers formed from either Aβ or amylin are capable of associating with lipid membranes, inducing perturbations of their integrity with the resultant deregulation of cellular metal ion homeostasis. One here probes the functional aspects of Aβ and amylin introduced above, using enantiopure materials and racemic mixtures in the biophysical setting. Catalytic production of reactive oxygen species may be investigated, with hydrogen peroxide production being a convenient readout, as it is readily detectable by fluorescent techniques. The ability of oligomeric assemblies, derived from Aβ or amylin, to bind to model lipid membranes and induce transmembrane calcium flow may be studied as a function of chirality. Interactions between amyloids and lipid bilayers are known to induce structural changes in amyloid aggregates. The structural approaches described above (microscopy in particular), can be employed to support functional findings regarding lipid bilayer disruption by amyloid oligomers.

### Example 4: The Chiral Slider Approach to Design D-Amyloid Polypeptides

Optimal binders of all-L Aβ and amylin may be obtained when only a subset of their constituent amino acids is subjected to chiral inversion, while the rest remains in the native all-L form (partial chirality inversion). To optimize for molecular recognition properties of the two polypeptides in focus, the corresponding analogues with partial chirality inversion are synthesized (FIG. 4A, 4B, the chiral slider approach). A sliding chiral frame of six amino acids is chosen as a reasonable starting point, because this is the characteristic length of dry interfaces, termed steric zippers, which are critical for amyloidogenic protein aggregation. Single chiral amino acid substitutions can also be performed. Hotspots of preferred chiral substitution identified using this method are combined into a matrix in order to identify the optimal combinations of D-amino acids integrated into Aβ and amylin, yielding the optimal chirality inversion fingerprint.

This method may be contrasted with that of Eisenberg, cited above. Eisenberg and co-workers used short peptides that cover steric zipper regions of diverse toxic amyloids, including Aβ and amylin, as an anti-aggregation strategy.

The present methods employ full-length amyloids and are directed to a pro-aggregation approach. That is, one uses the effects of a synthetic polypeptide that comprises or consists of a short stretch of D-amino acids to measure the aggregation of the synthetic polypeptide with the native amyloid polypeptide.

### Example 5: Scavenging Blood-Borne all-L-Aβ and Determining Ability of D-Isomers to Cross the Blood-Brain Barrier

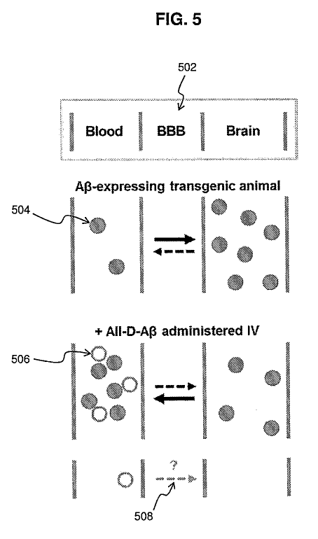

All-D-Aβ (a synthetic polypeptide) may be administered intravenously in a method to scavenge blood-borne all-L-Aβ. As schematically illustrated in FIG. 5, the blood compartment (left column) is separated from the brain compartment (right column) by the blood-brain barrier (BBB; center column). Transport of all-L-Aβ (closed circles) across the BBB into the brain is favored in an Aβ-expressing transgenic animal, but the balance may shift to export of all-L-Aβ out of the brain when all-D-Aβ (open circles) is administered intravenously, due to enhanced aggregation between the opposite enantiomers. Further, intravenous administration of all-D-Aβ allows one to test the ability of all-D-Aβ to be transported across the blood-brain barrier into the brain (bottom).

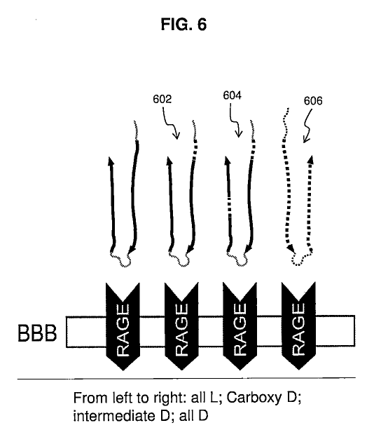

Referring now to FIG. 6, another mechanism for reducing the brain burden on L-Aβ utilizes a protein termed “RAGE.” Briefly, the method of “Receptor of advanced glycation end-products” (RAGE) is described in detail in Deamer et al. “RAGE mediates amyloid-beta peptide transport across the blood-brain barrier and accumulation in brain,” Nat. Med. 2003, 9(7):907-13.

As shown in FIG. 6, various constructs D-containing L-Aβ peptides 602 (D-carboxyl segment), 604(D-carboxyl segment and short N-region segment) and All-D A β (606) are prepared as described in connection with FIGS. 4A and 4B and administered to a subject, or added to an assay mixture containing RAGE receptors on a membrane, such that the translation into and/or through the blood brain barrier can be assessed in reference to different D-containing amyloid polypeptides. Using endogenous brain receptors of advanced glycosylation products, these constructs are transferred into the brain compartment using these specialized receptors in the blood-brain barrier. In animal studies, the various constructs are used to evaluate the transport characteristics of an engineered (all D- or partial D-) amyloid peptide. The import of the peptide into the brain is enabled by the receptor for advanced glycation end-products, whereas lipoprotein receptor proteins 1 and 2 are responsible for exporting it back into the blood stream. This leads to an equilibration between the two compartments. The transport of Aβ across the blood-brain barrier is likely to be stereospecific. Thus, it is of high interest, how much chiral substitution in the peptide can be tolerated before the brain import breaks down. An ex vivo model of the blood-brain barrier has been previously reported so that the use of experimental animals is not required. This convenient model system can allow one to tailor experiments to investigate the peptidic library, obtained through the chiral slider approach, with regard to transport across the blood-brain barrier, mediated by the receptor for advanced glycation end-products. Chirality can serve as a tool to study the function of the receptor. Furthermore, there is an independent, highly relevant biomedicinal aspect that emerges. Certain partial chiral mutants of Aβ may retain the ability to cross the blood-brain barrier, while losing the neuro- and synaptotoxicity associated with the all-L Aβ polypeptide. An Aβ analog that combined these two properties would possess the potential of giving rise to a fundamentally novel Trojan horse strategy for delivery of therapeutics into the brain. Comparisons with the amylin polypeptide may be performed in order to gain insight into target specificity of the receptor for advanced glycation end-products as a function of amyloid peptide composition.

### Example 6: A Mixture of L-Ab42 and D-Ab42 Forms a Precipitate, while an L-Ab42 Mixture Remains in Clear Solution

General Procedure for the Reconstitution of Amyloid Beta 42 (Aβ-42) for Biophysical Characterization

Commercially purchased all L-Aβ-42 (Aaptech) and all D-Aβ-42 (Aaptech) were treated with the appropriate volume of an aqueous solution of 0.1% NH₄OH in order to obtain a concentration of 1 mg/ml. All samples were analyzed by nanodrop to confirm their concentration and if required subjected to fractionation into the appropriate volume as required for the biophysical experiment. Following fractionation, the samples were incubated at room temperature for 90 minutes before being flash frozen in liquid nitrogen and placed on the lyophilizer overnight.

To reconstitute the protein for the biophysical technique, the pretreated 0.1% NH₄OH Aβ-42 was dissolved in a volume of 20 mM NaOH that represented 5% of the total volume required for the experiment. The solution was sonicated for 30 seconds and then diluted to the total volume using freshly prepared 20 mM phosphate buffer at pH 7.4. The phosphate buffer did not contain sodium chloride due to the absorption of the chloride ion at 190 nm interfering with circular dichroism (CD) experimental readings. In all instances the biophysical technique was started immediately following dissolution into the phosphate buffer.

### Dynamic Light Scattering (DLS) Analysis of Enantiopure and Racemate Mixtures of Amyloid Beta 42 (Aβ-42)

Dynamic light scattering experiments were performed on a BI-200 light scattering instrument (Brookhaven) using a laser source of λ=633 nm and a detector at a scattering angle of θ=90 degrees. Relaxation time distributions were obtained numerically from the field autocorrelation function g1(t). Particle size distributions were extracted from the latter through the Stokes-Einstein equation assuming spherical shapes of the particles. All samples were incubated at 37° C. using a temperature controlled oil bath and removed periodically every 24 hours for DLS analysis.

For experiments involving enantiopure Aβ-42, 3 mg of the NH₄OH pre-treated protein was dissolved in 150 μl of an aqueous solution of 20 mM NaOH and sonicated for 30 seconds. 2.85 ml of a freshly prepared 20 mM phosphate buffer not containing sodium chloride was then added and the mixture vortexed for 5 seconds. The solution was filtered through a 0.1 μm millipore durapore PVDF syringe filter and used immediately for analysis.

For investigations into racemate mixtures of the protein, 1.5 mg of all-L Aβ-42 and 1.5 mg of all-D Aβ-42 were pre-treated individually with 0.1% NH₄OH as outlined above. 75 μl of an aqueous solution of 20 mM NaOH were added to each pre-treated sample and sonicated for 30 seconds. 1.42 ml of freshly prepared 20 mM phosphate buffer not containing sodium chloride was then added to each sample and the two solutions combined and vortexed for 5 seconds to bring the total concentration of the solution to the same as the enantiopure experiments. The racemate mixture was filtered through a 0.1 μm millipore durapore PVDF syringe filter and used immediately for analysis.

Precipitation

Observation of the racemic and all-L Aβ-42 peptide revealed that the racemic mixture formed a precipitate after 24 hours, whereas the all-L counterpart remained in clear solution for at least 96 hours under identical conditions. A dramatic enhancement of aggregation was observed in the racemic amyloid mixture.

### Example 7: Pharmaceutical or Diagnostic Formulations for In Vivo Use

As discussed above, D-amino acid containing amyloid polypeptides have in vivo uses for tying up L-amyloid polypeptide (see Example 6), measuring L-amyloid polypeptide, or altering the balance of L-amyloid polypeptide in a blood or brain compartment due to translocation across the blood brain barrier.

Accordingly, there is provided a formulation for D-amino acid polypeptides, i.e., D-amyloid polypeptides that correspond in primary sequence to an L-amyloid polypeptide of interest.

According to the present invention, D-amyloid polypeptide may be formulated for use as a therapeutic agent when included in solution with agents that are effective in stabilizing the D-amyloid polypeptide against precipitation from solution, thermal degradation and adsorption. Such agents include non-ionic surfactants such a polysorbate-80, as well as propylene glycol, polyethylene glycol, lysine, arginine, cysteine, glutathione, ethanol and other alcohols. The preferred formulations of the present invention also may include other ingredients that function to improve the therapeutic capabilities of the D-amyloid polypeptide. Such other ingredients include sodium chloride, glycerol, human serum albumin, sodium phosphate, and tris(hydroxymethyl) aminomethane.

As used in the specification, “pharmaceutically effective amount” means an amount of D-amyloid polypeptide which is therapeutically effective in various administration regimes in the prevention and treatment of peripheral nerve damage. “Biologically acceptable” applies to materials characterized by the absence of significant adverse biological effects in vivo. “Room temperature” is between about 22° C. to about 25° C. “Body temperature” is between about 36° C. to about 40° C.

A further intrathecal formulation may be adapted from US 20080064725, “Intrathecal administration of triptan compositions to treat non-migraine pain.” A method for intracranial delivery may be adapted from “Drug Delivery device,” US 20130344125. A typical intravenous formulation may be adapted from U.S. Pat. No. 7,309,759, “Compositions and methods for treating infections using cationic peptides alone or in combination with antibiotics.”

Also, the present peptides may be delivered via oral administration, therapeutic proteins are almost exclusively administered by parenteral routes, such as intravenous (IV), subcutaneous (SC) or intramuscular (IM) injection. From the convenience standpoint, SC administration of therapeutic proteins is often a preferred route. In particular, the suitability of SC dosing for self-administration translates into significantly reduced treatment costs. Absorption of therapeutic proteins from the SC injection site tends to be slow compared to small molecules, and the absorption rates depend on the size of the molecule. See, Vugmeyster et al., “Pharmacokinetics and toxicology of therapeutic proteins: Advances and challenges” World J Biol Chem. 2012 Apr. 26; 3(4): 73-92. The concentration of a peptide stabilizer or mixtures thereof in a stable pharmaceutical composition described herein is between about 0.01 g/L and about 10 g/L. In another embodiment, the concentration of the peptide stabilizer is between about 0.5 g/L and about 5 g/L. In still another embodiment, the concentration of the peptide stabilizer is about 1 g/L.

In one embodiment of the present invention, the stable pharmaceutical protein composition contains a surfactant. While not being bound to a particular theory, it is believed that the presence of a surfactant in the solution reduces the adhesion of a biologically active protein, such as EPO to the walls of the container, in which the formulation is stored. The amount of surfactant used in the formulations described herein ranges from about 0.0005% w/v to about 0.5% w/v. In one embodiment, the amount of surfactant, particularly Tween® 80 is 0.03% w/v. In another embodiment, the surfactant is suitable for parenteral administration.

Any surfactant which is pharmaceutically acceptable can be included in the composition of the invention. Such surfactants include, without limitation, nonionic surfactants (e.g., polyoxyalkylene sorbitan fatty acid esters, sorbitan fatty acid esters, alkylene glycol fatty acid esters, polyoxyalkylene fatty acid esters, fatty acid esters, polyoxyalkylene fatty acid ethers, C16-C24 fatty acids, fatty acid mono-, di- or poly-glycerides, polyoxyalkylene alkyl phenols, alkyl phenyl ethers, polyoxyethylene polyoxypropylene block copolymers, fatty amine oxides, fatty acid alkanolamides, alkyl cellulose, carboxyalkyl cellulose, polyoxyalkylene castor oil derivatives), anionic surfactants (e.g., alkyl sulfates, olefin sulfates, ether sulfates, monoglyceride sulfates, alkyl sulfonates, aryl sulfonates, olefin sulfonates, alkyl sulfosuccinates, aryl sulfosuccinates, including sodium lauryl sulfate, dioctyl sodium sulfosuccinate and dioctyl sodium sulfonate), cationic surfactants (e.g., benzalkonium salts, polyoxyalkylene alkylamines, alkylamines, alkanolamine fatty acid esters, quaternary ammonium fatty acid esters, dialkyl ammonium salts, alkyl pyridinium salts including stearylamine, triethanolamine oleate, benzethonium chloride), amphoteric surfactants (e.g., alkyl β-aminopropionates, 2-alkylimidazoline quaternary ammonium salts) and zwitterionic surfactants. Nonionic surfactants for use in compositions of the invention include, but are not limited to, polyoxyethylene(20) sorbitan monolaurate (Tween® 20), polyoxyethylene(4) sorbitan monolaurate (Tween® 21), polyoxyethylene(20) sorbitan monopalmitate (Tween® 40), polyoxyethylene(20) sorbitan monostearate (Tween® 60), polyoxyethylene(20) sorbitan tristearate (Tween® 65), polyoxyethylene(20) sorbitan monooleate (Tween® 80 or polysorbate 80), or polyoxyethylene(20) sorbitan trioleate (Tween® 85), lauromacrogol 400, polyoxyl 40 stearate, polyoxyethylene hydrogenated castor oil 10, 50 and 60, glycerol monostearate, glycerol monooleate, polysorbate 40, 60, 65 and 80, sucrose fatty acid ester, sorbitan laurate, sorbitan oleate, sorbitan palmitate, sorbitan stearate, sorbitan tristearate, sorbitan sesquioleate, sorbitan trioleate, sorbitan isostearate, propylene glycol monostearate, polyoxyethylene monostearate, polyoxyethylene distearate, glyceryl monostearate, polyoxyethylene lauryl ether, polyoxyethylene cetyl ether, polyoxyethylene stearyl ether, polyoxyethylene oleyl ether, palmitic acid, stearic acid, oleic acid, ethyl oleate, isopropyl myristate, sodium palmitate, sodium stearate, sodium oleate, nonylphenol polyethoxyethanols, tributylphenoxy-polyethoxyethanol, octylphenoxy-polyethoxyethanol, polyoxyethylene glycerol triricinoleate or polyoxyl 35 castor oil (Cremophor® EL, BASF Corp.), polyoxyethylene glycerol oxystearate (Cremophor® RH 40), polyethylene glycol 60 hydrogenated castor oil (Cremophor® RH 60), Poloxamer® 124, Poloxamer® 188, Poloxamer® 237, Poloxamer® 388, Poloxamer® 407 (BASF Wyandotte Corp.), methylcellulose and carboxymethyl cellulose. Preferably, the surfactant used is a polyoxyethylene sorbitan mono- or tri-lauryl, palmityl, stearyl or oleyl ester such as polyoxyethylene(20) sorbitan monolaurate (Tween® 20), polyoxyethylene(4) sorbitan monolaurate (Tween® 21), polyoxyethylene(20) sorbitan monopalmitate (Tween® 40), polyoxyethylene(20) sorbitan monostearate (Tween® 60), polyoxyethylene(20) sorbitan tristearate (Tween® 65), polyoxyethylene(20) sorbitan monooleate (Tween® 80 or polysorbate 80), or polyoxyethylene(20) sorbitan trioleate (Tween® 85).

Optionally, the stable pharmaceutical protein formulations described herein may include preservatives, buffering agents, isotonicity agents, and other conventional components used in formulating pharmaceutical compositions.

### Example 8: Fibril Formation is Accelerated in Racemic Ab

The rate of fibril formation was investigated by Thioflavin T (ThT) assay for all-L-Aβ, all-D-Aβ, and racemic Aβ. In this particular example, Aβ40 was investigated. For all-L-Aβ40WT, 100 μg of all-L-Aβ40WT were dissolved in 50 μl of 20 mM NaOH and then 950 μl of 20 uM ThT were added (20 μM of ThT in 1×PBS). 200 μl of this solution was used in each well and fluorescence kinetics (excitation 440 emission 485) at 37° C. with shaking was measured. The conditions for all-D-Aβ40WT were the same as those for all-L-Aβ40WT. For the racemate, 50 μg of L-Aβ40 dissolved in 250 of 20 mM NaOH was mixed with 50 μg of D-Aβ40, also dissolved in 25 μl of NaOH, obtaining 50 μl of racemate mixture. 950 μl of 20 μM of ThT were added and then 200 μl were used in each well to measure fluorescence at 37° C.

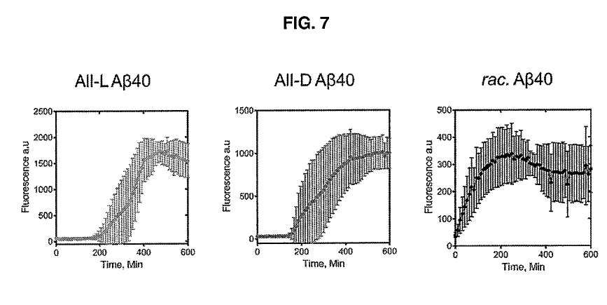

The ThT curves for all-L-Aβ40 and all-D-Aβ40 were similar (FIG. 7). For both cases, the lag time was around 200 minutes, reaching the maximum fluorescence for fibril formation at around 400 minutes. Racemic Aβ40, however, showed fibril formation at 0 time, reaching its maximum value at 200 minutes (FIG. 7), indicating a dramatic acceleration on fibril formation for racemic Aβ40. It is expected that a racemic mixture of any of the Aβ peptides (e.g., any of Aβ36 to Aβ49) will exhibit a similar acceleration towards fibril formation.

### Example 9: Fibril Morphology for Racemic Ab

The fibril morphology for racemic Aβ was investigated. In this particular example, Aβ40 was investigated. 3 μl of ThT sample for all-L-Aβ40, all-D-Aβ40, and the racemate were added to a grid. After 1 minute, the solution was wiped, then the grid was washed with 1% uranyl acetate and air dried. The three samples were grown at 1:1 peptide:Tht concentration ratio. Images were captured using a Tecnai 12 TEM instrument.

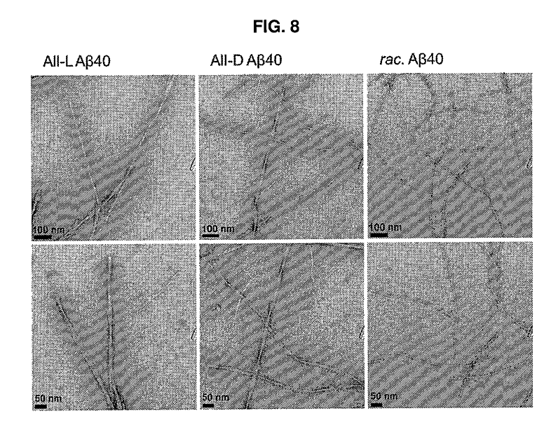

As shown in FIG. 8, the fibers for all-L-Aβ40 and all-D-Aβ40 showed similar length and diameter. Both fibers showed helicity, appearing to be of inverted helicity for all-L-Aβ40 compared to all-D-Aβ40. The fibers for racemic Aβ40 are smaller and thinner as compared to all-L-Aβ40 compared to all-D-Aβ40, with no apparent helicity. It is expected that a racemic mixture of any of Aβ36 to Aβ49 will exhibit fibers of similar morphology.

### Example 10: Shift Toward Formation of Higher Molecular Weight Aggregates in Racemic Ab

PICUP (Photo-Induced Cross-Linking of Unmodified Proteins) was used to determine the population of low molecular weight oligomers of the amyloid beta peptide. These soluble oligomers are believed to serve as a basis for the aggregation of the peptide. The technique involves covalently cross-linking aggregate states of the peptide that occur in solution which would otherwise be unobtainable with traditional electrophoresis due to SDS disruption of these aggregates.

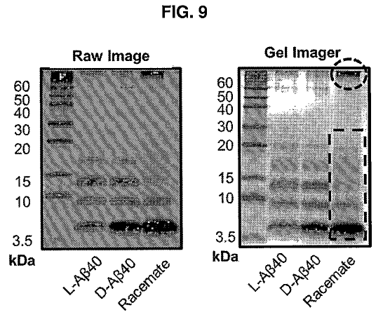

Results are shown in FIG. 9, which shows a raw image of a gel (left image) and gel imager image of the gel (right) that includes, from left to right, a size marker, L-Aβ40 alone, D-Aβ40 alone, and racemic Aβ40. As expected, the two individual enantiomers of amyloid beta behave identical to one and other. These results are shown in the first two lanes of the gel and show that, in solution, the peptide exists in numerous oligomeric states ranging from a monomer (lowest band on the gel) to a pentamer and hexamer (the two bands found around the 20 kDa reference band). However, in the racemic mixture of the peptide, a distinct change in the population states is observed. The lower bands (monomer through pentamer/hexamer) are reduced in their density and therefore indicate a significant reduction in the degree in which the peptide adopts these population states (indicated in the dashed box on the gel imager image). Furthermore, a second band appears at the top of the lane (indicated in the dashed circle on the gel imager image) which is far larger (>60 kDa) than any aggregates observed when only a single enantiomer is present. This indicates that, in the racemate sample, a shift occurs from production of low molecular weight amyloid beta oligomers to the formation of higher molecular weight peptide aggregates.

Notwithstanding the appended clauses, the disclosure set forth herein is also defined by the following clauses:

1. A pharmaceutical formulation, comprising:
an amyloid polypeptide comprising at least one D-amino acid; and
a pharmaceutically acceptable carrier.
   2. The pharmaceutical formulation of Clause 1, wherein the amyloid polypeptide comprising at least one D-amino acid is a β-Amyloid (Aβ) polypeptide, a Type 2 diabetes (amylin) amyloid polypeptide, an Alpha-synuclein (SNCA) amyloid polypeptide, a transthyretin (TTR) polypeptide, a Huntingtin polypeptide, or a fragment thereof.
   3. The pharmaceutical formulation of Clause 2, wherein the amyloid polypeptide comprising at least one D-amino acid comprises 100% amino acid sequence identity to a wild-type β-Amyloid polypeptide, a wild-type Type 2 diabetes (amylin) amyloid polypeptide, a wild-type Alpha-synuclein (SNCA) amyloid polypeptide, a wild-type transthyretin (TTR) polypeptide, a wild-type Huntingtin polypeptide, or a fragment thereof.
   4. The pharmaceutical formulation of any one of Clauses 1 to 3, wherein the amyloid polypeptide comprising at least one D-amino acid is an Aβ polypeptide of from 36 to 49 amino acids in length.
   5. The pharmaceutical formulation of Clause 4, wherein the Aβ polypeptide comprising at least one D-amino acid is an Aβ42 polypeptide.
   6. The pharmaceutical formulation of Clause 4, wherein the Aβ polypeptide comprising at least one D-amino acid is an Aβ40 polypeptide.
   7. The pharmaceutical formulation of any one of Clauses 1 to 6, wherein the amyloid polypeptide comprising at least one D-amino acid comprises 2 or more D-amino acids.
   8. The pharmaceutical formulation of any one of Clauses 1 to 6, wherein the amyloid polypeptide comprising at least one D-amino acid comprises 25% or more D-amino acids.
   9. The pharmaceutical formulation of any one of Clauses 1 to 6, wherein the amyloid polypeptide comprising at least one D-amino acid comprises 50% or more D-amino acids.
   10. The pharmaceutical formulation of any one of Clauses 1 to 6, wherein the amyloid polypeptide comprising at least one D-amino acid comprises 75% or more D-amino acids.
   11. The pharmaceutical formulation of any one of Clauses 1 to 6, wherein the amyloid polypeptide comprising at least one D-amino acid comprises 90% or more D-amino acids.
   12. The pharmaceutical formulation of any one of Clauses 1 to 6, wherein each amino acid of the amyloid polypeptide comprising at least one D-amino acid is a D-amino acid.
   13. A kit, comprising:
the pharmaceutical formulation of any one of Clauses 1 to 12.
   14. The kit of Clause 13, wherein the kit comprises the pharmaceutical formulation in one or more unit dosages.
   15. The kit of Clause 13 or Clause 14, further comprising instructions for using the formulation to treat an individual in need thereof.
   16. A method comprising administering a therapeutically effective amount of the pharmaceutical formulation of any one of Clauses 1 to 12 to an individual in need thereof.
   17. The method according to Clause 16, wherein the administering is by intrathecal, intracranial, or intravenous administration.
   18. The method according to Clause 16 or Clause 17, wherein the individual in need thereof has Alzheimer's Disease (AD), and the amyloid polypeptide comprising at least one D-amino acid is an Aβ polypeptide of from 36 to 49 amino acids in length.
   19. The method according to Clause 18, wherein the amyloid polypeptide comprising at least one D-amino acid is an Aβ42 polypeptide.
   20. A method of forming racemic amyloid polypeptide aggregates, comprising:
contacting aggregates comprising all-L amyloid polypeptides with amyloid polypeptides comprising at least one D-amino acid, wherein the amyloid polypeptides comprising at least one D-amino acid correspond to the all-L amyloid polypeptides,
to form racemic amyloid polypeptide aggregates.
   21. The method according to Clause 20, wherein the contacting comprises combining the aggregates comprising all-L amyloid polypeptides and the amyloid polypeptides comprising at least one D-amino acid under aggregation conditions in a container.
   22. The method according to Clause 21, wherein the container is a tube or a well of a plate.
   23. The method according to Clause 20, wherein the contacting occurs in vivo.
   24. The method according to Clause 23, wherein the contacting comprises administering the amyloid polypeptides comprising at least one D-amino acid to an individual comprising the aggregates comprising all-L amyloid polypeptides.
   25. The method according to Clause 24, wherein the administering comprises administering the amyloid polypeptides comprising at least one D-amino acid to the individual via intrathecal, intracranial, or intravenous administration.
   26. The method according to any one of Clauses 20 to 25, wherein the aggregates comprising all-L amyloid polypeptides comprise all-L β-Amyloid (Aβ) polypeptides, and the amyloid polypeptides comprising at least one D-amino acid are Aβ polypeptides comprising at least one D-amino acid.
   27. The method according to Clause 26, wherein the Aβ polypeptides comprising at least one D-amino acid are Aβ polypeptides of from 36 to 49 amino acids in length.
   28. The method according to Clause 26, wherein the Aβ polypeptides comprising at least one D-amino acid are Aβ40 polypeptides.
   29. The method according to Clause 26, wherein the Aβ polypeptides comprising least one D-amino acid are Aβ42 polypeptides.
   30. The method according to any one of Clauses 20 to 29, wherein the amyloid polypeptides comprising at least one D-amino acid comprise 2 or more D-amino acids.
   31. The method according to any one of Clauses 20 to 29, wherein the amyloid polypeptides comprising at least one D-amino acid comprise 25% or more D-amino acids.
   32. The method according to any one of Clauses 20 to 29, wherein the amyloid polypeptides comprising at least one D-amino acid comprise 50% or more D-amino acids.
   33. The method according to any one of Clauses 20 to 29, wherein the amyloid polypeptides comprising at least one D-amino acid comprise 75% or more D-amino acids.
   34. The method according to any one of Clauses 20 to 29, wherein the amyloid polypeptides comprising at least one D-amino acid comprise 90% or more D-amino acids.
   35. The method according to any one of Clauses 20 to 29, wherein each amino acid of the amyloid polypeptides comprising at least one D-amino acid is a D-amino acids.
   36. A method for reducing solubility of an all L-amyloid polypeptide in a fluid, comprising:
(a) contacting the all L-amyloid polypeptide in a fluid with a synthetic polypeptide having an amino acid sequence essentially identical to the all L-amyloid polypeptide, except that it contains D-amino acids, and
(b) incubating the all-L amyloid polypeptide with the synthetic polypeptide under conditions in which a complex comprising the all-L amyloid polypeptide and the synthetic polypeptide is formed,
whereby the complex has a solubility less than a complex formed of all L-amyloid polypeptides.
   37. The method according to Clause 36, wherein the complex contains at least one of 10%, 20%, 30%, 40%, or 50% synthetic polypeptide, the remainder being all-L amyloid polypeptide; and/or the complex contains a synthetic polypeptide consisting essentially of all D-amino acids.
   38. The method according to Clause 36, wherein the all-L amyloid polypeptide is a native amyloid polypeptide, and the fluid is blood or cerebral spinal fluid.
   39. The method according to Clause 36, further comprising removing the complex from the fluid.
   40. The method according to Clause 39, wherein the fluid is from a host organism, and depleted fluid from the step of removing the complex is returned to the host.
   41. The method according to Clause 36, wherein the step of incubating the all-L amyloid polypeptide with the synthetic polypeptide further comprises the step of immobilizing the synthetic polypeptide on a plurality of beads and mixing the beads with the fluid.
   42. The method according to Clause 41, wherein the beads are magnetic beads and are removed from the fluid by a magnetic material mixed with the fluid and removed from the fluid.
   43. The method according to Clause 36, wherein the amyloid polypeptide is essentially identical to a polypeptide selected from the group consisting of: a β-Amyloid (Aβ) polypeptide, a Type 2 diabetes (amylin) amyloid polypeptide, an Alpha-synuclein (SNCA) amyloid polypeptide, a transthyretin (TTR) polypeptide, a Huntingtin polypeptide, or a fragment thereof.
   44. A method for characterizing an amyloid polypeptide of interest, comprising:
(a) contacting the amyloid polypeptide of interest with a second amyloid polypeptide comprising D-amino acids and having an essentially identical sequence to the amyloid polypeptide of interest;
(b) forming an aggregate between the amyloid polypeptide of interest with the second amyloid polypeptide; and
(c) measuring the amount of aggregation that has formed.
   45. The method according to Clause 44, wherein the measuring is done with a nuclear magnetic resonance (NMR) spectroscopic technique and wherein the amyloid polypeptide of interest and the second amyloid polypeptide comprising D-amino acids are chemically linked to different NMR labels.
   46. The method according to Clause 44, wherein the amyloid polypeptide of interest is selected from the group consisting of: a β-Amyloid (Aβ) polypeptide, a Type 2 diabetes (amylin) amyloid polypeptide, an Alpha-synuclein (SNCA) amyloid polypeptide, a transthyretin (TTR) polypeptide, a Huntingtin polypeptide, or a fragment thereof.
   47. A method for removing an amyloid polypeptide from a bodily fluid, comprising contacting the bodily fluid with a synthetic polypeptide substantially identical in sequence to the amyloid polypeptide, but comprising at least a segment thereof of L-amino acids, wherein the synthetic polypeptide is immobilized to permit removal of the synthetic polypeptide in the form of a complex, the method further comprising forming the complex between the amyloid polypeptide in the synthetic polypeptide which permits removal of the complex from the bodily fluid.
   48. The method according to Clause 47, wherein the synthetic polypeptide is immobilized on a bead.
   49. A synthetic amyloid polypeptide comprising a portion of L-amino acids and at least one portion of D-amino acids, where the portion of D-amino acids exhibits a higher affinity for a counterpart amyloid peptide than a corresponding portion of L-amino acids.
   50. The synthetic amyloid polypeptide of Clause 49, selected from the group consisting of: a β-Amyloid (Aβ) polypeptide, a Type 2 diabetes (amylin) amyloid polypeptide, an Alpha-synuclein (SNCA) amyloid polypeptide, a transthyretin (TTR) polypeptide, a Huntingtin polypeptide, or a fragment thereof.
   51. The synthetic amyloid polypeptide of Clause 49 or Clause 50, comprising a portion of L-amino acids and a contiguous stretch of 5-15 D-amino acids.
   52. A pharmaceutical formulation, comprising:
the synthetic amyloid polypeptide of any one of Clauses 49 to 51; and
a pharmaceutically acceptable carrier.
   53. A method for identifying an amyloid polypeptide binding compound, comprising:
(a) providing a solution containing an amyloid polypeptide of interest;
(b) adding to the mixture a second amyloid polypeptide having an essentially identical sequence to the amyloid polypeptide of interest, further having a defined portion of 1 to 10 D-amino acids and measuring aggregation formation;
(c) repeating step (b) with a third amyloid polypeptide having an essentially identical sequence to the amyloid polypeptide of interest, further having a defined portion of 1 to 10 D-amino acids adjacent to the defined portion in step (b); and
(d) comparing results of aggregation formation in steps (b) and (c).

### CONCLUSION

The above specific description is meant to exemplify and illustrate the invention and should not be seen as limiting the scope of the invention, which is defined by the literal and equivalent scope of the appended claims. Any patents or publications mentioned in this specification are intended to convey details of methods and materials useful in carrying out certain aspects of the invention which may not be explicitly set out but which would be understood by workers in the field. Such patents or publications are hereby incorporated by reference to the same extent as if each was specifically and individually incorporated by reference and contained herein, as needed for the purpose of describing and enabling the method or material referred to.

What is claimed is:

1. A pharmaceutical formulation, comprising:
an amyloid polypeptide comprising at least one D-amino acid; and
a pharmaceutically acceptable carrier.

2. The pharmaceutical formulation of claim 1, wherein the amyloid polypeptide comprising at least one D-amino acid is a β-Amyloid (Aβ) polypeptide, a Type 2 diabetes (amylin) amyloid polypeptide, an Alpha-synuclein (SNCA) amyloid polypeptide, a transthyretin (TTR) polypeptide, a Huntingtin polypeptide, or a fragment thereof.

3. The pharmaceutical formulation of claim 2, wherein the amyloid polypeptide comprising at least one D-amino acid comprises 100% amino acid sequence identity to a wild-type β-Amyloid polypeptide, a wild-type Type 2 diabetes (amylin) amyloid polypeptide, a wild-type Alpha-synuclein (SNCA) amyloid polypeptide, a wild-type transthyretin (TTR) polypeptide, a wild-type Huntingtin polypeptide, or a fragment thereof.

4. The pharmaceutical formulation of any one of claims 1 to 3, wherein the amyloid polypeptide comprising at least one D-amino acid is an Aβ polypeptide of from 36 to 49 amino acids in length.

5. The pharmaceutical formulation of claim 4, wherein the Aβ polypeptide comprising at least one D-amino acid is an Aβ42 polypeptide.

6. The pharmaceutical formulation of claim 4, wherein the Aβ polypeptide comprising at least one D-amino acid is an Aβ40 polypeptide.

7. The pharmaceutical formulation of any one of claims 1 to 6, wherein the amyloid polypeptide comprising at least one D-amino acid comprises 2 or more D-amino acids.

8. The pharmaceutical formulation of any one of claims 1 to 6, wherein the amyloid polypeptide comprising at least one D-amino acid comprises 25% or more D-amino acids.

9. The pharmaceutical formulation of any one of claims 1 to 6, wherein the amyloid polypeptide comprising at least one D-amino acid comprises 50% or more D-amino acids.

10. The pharmaceutical formulation of any one of claims 1 to 6, wherein the amyloid polypeptide comprising at least one D-amino acid comprises 75% or more D-amino acids.

11. The pharmaceutical formulation of any one of claims 1 to 6, wherein the amyloid polypeptide comprising at least one D-amino acid comprises 90% or more D-amino acids.

12. The pharmaceutical formulation of any one of claims 1 to 6, wherein each amino acid of the amyloid polypeptide comprising at least one D-amino acid is a D-amino acid.

13. A kit, comprising:
the pharmaceutical formulation of any one of claims 1 to 12.

14. The kit of claim 13, wherein the kit comprises the pharmaceutical formulation in one or more unit dosages.

15. The kit of claim 13 or claim 14, further comprising instructions for using the formulation to treat an individual in need thereof.

16. A method comprising administering a therapeutically effective amount of the pharmaceutical formulation of any one of claims 1 to 12 to an individual in need thereof.

17. The method according to claim 16, wherein the administering is by intrathecal, intracranial, or intravenous administration.

18. The method according to claim 16 or claim 17, wherein the individual in need thereof has Alzheimer's Disease (AD), and the amyloid polypeptide comprising at least one D-amino acid is an Aβ polypeptide of from 36 to 49 amino acids in length.

19. The method according to claim 18, wherein the amyloid polypeptide comprising at least one D-amino acid is an Aβ42 polypeptide.

20. A method of forming racemic amyloid polypeptide aggregates, comprising:
contacting aggregates comprising all-L amyloid polypeptides with amyloid polypeptides comprising at least one D-amino acid, wherein the amyloid polypeptides comprising at least one D-amino acid correspond to the all-L amyloid polypeptides,
to form racemic amyloid polypeptide aggregates.

21. The method of claim 20, wherein the contacting comprises combining the aggregates comprising all-L amyloid polypeptides and the amyloid polypeptides comprising at least one D-amino acid under aggregation conditions in a container.

22. The method of claim 21, wherein the container is a tube or a well of a plate.

23. The method of claim 20, wherein the contacting occurs in vivo.

24. The method of claim 23, wherein the contacting comprises administering the amyloid polypeptides comprising at least one D-amino acid to an individual comprising the aggregates comprising all-L amyloid polypeptides.

25. The method of claim 24, wherein the administering comprises administering the amyloid polypeptides comprising at least one D-amino acid to the individual via intrathecal, intracranial, or intravenous administration.

26. The method of any one of claims 20 to 25, wherein the aggregates comprising all-L amyloid polypeptides comprise all-L β-Amyloid (Aβ) polypeptides, and the amyloid polypeptides comprising at least one D-amino acid are Aβ polypeptides comprising at least one D-amino acid.

27. The method of claim 26, wherein the Aβ polypeptides comprising at least one D-amino acid are Aβ polypeptides of from 36 to 49 amino acids in length.

28. The method of claim 26, wherein the Aβ polypeptides comprising at least one D-amino acid are Aβ40 polypeptides.

29. The method of claim 26, wherein the Aβ polypeptides comprising least one D-amino acid are Aβ42 polypeptides.

30. The method according to any one of claims 20 to 29, wherein the amyloid polypeptides comprising at least one D-amino acid comprise 2 or more D-amino acids.

31. The method according to any one of claims 20 to 29, wherein the amyloid polypeptides comprising at least one D-amino acid comprise 25% or more D-amino acids.

32. The method according to any one of claims 20 to 29, wherein the amyloid polypeptides comprising at least one D-amino acid comprise 50% or more D-amino acids.

33. The method according to any one of claims 20 to 29, wherein the amyloid polypeptides comprising at least one D-amino acid comprise 75% or more D-amino acids.

34. The method according to any one of claims 20 to 29, wherein the amyloid polypeptides comprising at least one D-amino acid comprise 90% or more D-amino acids.

35. The method according to any one of claims 20 to 29, wherein each amino acid of the amyloid polypeptides comprising at least one D-amino acid is a D-amino acids.

36. A method for reducing solubility of an all L-amyloid polypeptide in a fluid, comprising:
(a) contacting the all L-amyloid polypeptide in a fluid with a synthetic polypeptide having an amino acid sequence essentially identical to the all L-amyloid polypeptide, except that it contains D-amino acids, and
(b) incubating the all-L amyloid polypeptide with the synthetic polypeptide under conditions in which a complex comprising the all-L amyloid polypeptide and the synthetic polypeptide is formed,
whereby the complex has a solubility less than a complex formed of all L-amyloid polypeptides.

37. A method for characterizing an amyloid polypeptide of interest, comprising:
(a) contacting the amyloid polypeptide of interest with a second amyloid polypeptide comprising D-amino acids and having an essentially identical sequence to the amyloid polypeptide of interest;
(b) forming an aggregate between the amyloid polypeptide of interest with the second amyloid polypeptide; and
(c) measuring the amount of aggregation that has formed.

38. A method for removing an amyloid polypeptide from a bodily fluid, comprising contacting the bodily fluid with a synthetic polypeptide substantially identical in sequence to the amyloid polypeptide, but comprising at least a segment thereof of L-amino acids, wherein the synthetic polypeptide is immobilized to permit removal of the synthetic polypeptide in the form of a complex, the method further comprising forming the complex between the amyloid polypeptide in the synthetic polypeptide which permits removal of the complex from the bodily fluid.

39. The method of claim 38, wherein the synthetic polypeptide is immobilized on a bead.

40. A synthetic amyloid polypeptide comprising a portion of L-amino acids and at least one portion of D-amino acids, where the portion of D-amino acids exhibits a higher affinity for a counterpart amyloid peptide than a corresponding portion of L-amino acids.

41. A pharmaceutical formulation, comprising:
the synthetic amyloid polypeptide of claim 40; and
a pharmaceutically acceptable carrier.

42. A method for identifying an amyloid polypeptide binding compound, comprising:
(a) providing a solution containing an amyloid polypeptide of interest;
(b) adding to the mixture a second amyloid polypeptide having an essentially identical sequence to the amyloid polypeptide of interest, further having a defined portion of 1 to 10 D-amino acids and measuring aggregation formation;
(c) repeating step (b) with a third amyloid polypeptide having an essentially identical sequence to the amyloid polypeptide of interest, further having a defined portion of 1 to 10 D-amino acids adjacent to the defined portion in step (b); and
(d) comparing results of aggregation formation in steps (b) and (c).


SEQUENCE LISTING (9 sequences)

SEQ ID NO: 1 - protein, 49 aa, Homo sapiens
DAEFRHDSGYEVHHQKLVFFAEDVGSNKGAIIGLMVGGVVIATVIVITL

SEQ ID NO: 2 - protein, 42 aa, Homo sapiens [sequence omitted]

SEQ ID NO: 3 - protein, 40 aa, Homo sapiens
DAEFRHDSGYEVHHQKLVFFAEDVGSNKGAIIGLMVGGVV

SEQ ID NO: 4 - protein, 89 aa, Homo sapiens
MGILKLQVFLIVLSVALNHLKATPIESHQVEKRKCNTATCATQRLANFLVHSSNNFGAILSSTNVGSNTYGKRNAVEVLKREPLNYLPL

SEQ ID NO: 5 - protein, 140 aa, Homo sapiens
MDVFMKGLSKAKEGVVAAAEKTKQGVAEAAGKTKEGVLYVGSKTKEGVVHGVATVAEKTKEQVTNVGGAVVTGVTAVAQKTVEGAGSIAAATGFVKKDQLGKNEEGAPQEGILEDMPVDPDNEAYEMPSEEGYQDYEPEA

SEQ ID NO: 6 - protein, 3145 aa, Homo sapiens
MATLEKLMKAFESLKSFQQQQQQQQQQQQQQQQQQQQQQQPPPPPPPPPPPQLPQPPPQAQPLLPQPQPPPPPPPPPPGPAVAEEPLHRPKKELSATKKDRVNHCLTICENIVAQSVRNSPEFQKLLGIAMELFLLCSDDAESDVRMVADECLNKVIKALMDSNLPRLQLELYKEIKKNGAPRSLRAALWRFAELAHLVRPQKCRPYLVNLLPCLTRTSKRPEESVQETLAAAVPKIMASFGNFANDNEIKVLLKAFIANLKSSSPTIRRTAAGSAVSICQHSRRTQYFYSWLLNVLLGLLVPVEDEHSTLLILGVLLTLRYLVPLLQQQVKDTSLKGSFGVTRKEMEVSPSAEQLVQVYELTLHHTQHQDHNVVTGALELLQQLFRTPPPELLQTLTAVGGIGQLTAAKEESGGRSRSGSIVELIAGGGSSCSPVLSRKQKGKVLLGEEEALEDDSESRSDVSSSALTASVKDEISGELAASSGVSTPGSAGHDIITEQPRSQHTLQADSVDLASCDLTSSATDGDEEDILSHSSSQVSAVPSDPAMDLNDGTQASSPISDSSQTTTEGPDSAVTPSDSSEIVLDGTDNQYLGLQIGQPQDEDEEATGILPDEASEAFRNSSMALQQAHLLKNMSHCRQPSDSSVDKFVLRDEATEPGDQENKPCRIKGDIGQSTDDDSAPLVHCVRLLSASFLLTGGKNVLVPDRDVRVSVKALALSCVGAAVALHPESFFSKLYKVPLDTTEYPEEQYVSDILNYIDHGDPQVRGATAILCGTLICSILSRSRFHVGDWMGTIRTLTGNTFSLADCIPLLRKTLKDESSVTCKLACTAVRNCVMSLCSSSYSELGLQLIIDVLTLRNSSYWLVRTELLETLAEIDFRLVSFLEAKAENLHRGAHHYTGLLKLQERVLNNVVIHLLGDEDPRVRHVAAASLIRLVPKLFYKCDQGQADPVVAVARDQSSVYLKLLMHETQPPSHFSVSTITRIYRGYNLLPSITDVTMENNLSRVIAAVSHELITSTTRALTFGCCEALCLLSTAFPVCIWSLGWHCGVPPLSASDESRKSCTVGMATMILTLLSSAWFPLDLSAHQDALILAGNLLAASAPKSLRSSWASEEEANPAATKQEEVWPALGDRALVPMVEQLFSHLLKVINICAHVLDDVAPGPAIKAALPSLTNPPSLSPIRRKGKEKEPGEQASVPLSPKKGSEASAASRQSDTSGPVTTSKSSSLGSFYHLPSYLKLHDVLKATHANYKVTLDLQNSTEKFGGFLRSALDVLSQILELATLQDIGKCVEEILGYLKSCFSREPMMATVCVQQLLKTLFGTNLASQFDGLSSNPSKSQGRAQRLGSSSVRPGLYHYCFMAPYTHFTQALADASLRNMVQAEQENDTSGWFDVLQKVSTQLKTNLTSVTKNRADKNAIHNHIRLFEPLVIKALKQYTTTTCVQLQKQVLDLLAQLVQLRVNYCLLDSDQVFIGFVLKQFEYIEVGQFRESEAIIPNIFFFLVLLSYERYHSKQIIGIPKIIQLCDGIMASGRKAVTHAIPALQPIVHDLFVLRGTNKADAGKELETQKEVVVSMLLRLIQYHQVLEMFILVLQQCHKENEDKWKRLSRQIADIILPMLAKQQMHIDSHEALGVLNTLFEILAPSSLRPVDMLLRSMFVTPNTMASVSTVQLWISGILAILRVLISQSTEDIVLSRIQELSFSPYLISCTVINRLRDGDSTSTLEEHSEGKQIKNLPEETFSRFLLQLVGILLEDIVTKQLKVEMSEQQHTFYCQELGTLLMCLIHIFKSGMFRRITAAATRLFRSDGCGGSFYTLDSLNLRARSMITTHPALVLLWCQILLLVNHTDYRWWAEVQQTPKRHSLSSFTKLLSPQMSGEEEDSDLAAKLGMCNREIVRRGALILFCDYVCQNLHDSEHLTWLIVNHIQDLISLSHEPPVQDFISAVHRNSAASGLFIQAIQSRCENLSTPTMLKKTLQCLEGIHLSQSGAVLTLYVDRLLCTPFRVLARMVDILACRRVEMLLAANLQSSMAQLPMEELNRIQEYLQSSGLAQRHQRLYSLLDRFRLSTMQDSLSPSPPVSSHPLDGDGHVSLETVSPDKDWYVHLVKSQCWTRSDSALLEGAELVNRIPAEDMNAFMMNSEFNLSLLAPCLSLGMSEISGGQKSALFEAAREVTLARVSGTVQQLPAVHHVFQPELPAEPAAYWSKLNDLFGDAALYQSLPTLARALAQYLVVVSKLPSHLHLPPEKEKDIVKFVVATLEALSWHLIHEQIPLSLDLQAGLDCCCLALQLPGLWSVVSSTEFVTHACSLIYCVHFILEAVAVQPGEQLLSPERRTNTPKAISEEEEEVDPNTQNPKYITAACEMVAEMVESLQSVLALGHKRNSGVPAFLTPLLRNIIISLARLPLVNSYTRVPPLVWKLGWSPKPGGDFGTAFPEIPVEFLQEKEVFKEFIYRINTLGWTSRTQFEETWATLLGVLVTQPLVMEQEESPPEEDTERTQINVLAVQAITSLVLSAMTVPVAGNPAVSCLEQQPRNKPLKALDTRFGRKLSIIRGIVEQEIQAMVSKRENIATHHLYQAWDPVPSLSPATTGALISHEKLLLQINPERELGSMSYKLGQVSIHSVWLGNSITPLREEEWDEEEEEEADAPAPSSPPTSPVNSRKHRAGVDIHSCSQFLLELYSRWILPSSSARRTPAILISEVVRSLLVVSDLFTERNQFELMYVTLTELRRVHPSEDEILAQYLVPATCKAAAVLGMDKAVAEPVSRLLESTLRSSHLPSRVGALHGVLYVLECDLLDDTAKQLIPVISDYLLSNLKGIAHCVNIHSQQHVLVMCATAFYLIENYPLDVGPEFSASIIQMCGVMLSGSEESTPSIIYHCALRGLERLLLSEQLSRLDAESLVKLSVDRVNVHSPHRAMAALGLMLTCMYTGKEKVSPGRTSDPNPAAPDSESVIVAMERVSVLFDRIRKGFPCEARVVARILPQFLDDFFPPQDIMNKVIGEFLSNQQPYPQFMATVVYKVFQTLHSTGQSSMVRDWVMLSLSNFTQRAPVAMATWSLSCFFVSASTSPWVAAILPHVISRMGKLEQVDVNLFCLVATDFYRHQIEEELDRRAFQSVLEVVAAPGSPYHRLLTCLRNVHKVTTC

SEQ ID NO: 7 - protein, 218 aa, Homo sapiens
MATLEKLMKAFESLKSFQQQQQQQQQQQQQQQQQQQQQQQPPPPPPPPPPPQLPQPPPQAQPLLPQPQPPPPPPPPPPGPAVAEEPLHRPKKELSATKKDRVNHCLTICENIVAQSVRNSPEFQKLLGIAMELFLLCSDDAESDVRMVADECLNKVIKALMDSNLPRLQLELYKEIKKNGAPRSLRAALWRFAELAHLVRPQKCRPYLVNLLPCLTRT

SEQ ID NO: 8 - protein, 126 aa, Homo sapiens
GPTGTGESKCPLMVKVLDAVRGSPAINVAVHVFRKAADDTWEPFASGKTSESGELHGLTEEEFVEGIYKVEIDTKSYWKALGISPFHEHAEVVFTANDSGPRRYTIAALLSPYSYSTTAVVTNPKE

SEQ ID NO: 9 - protein, 42 aa, Homo sapiens
DAEFRHDSGYEVHAQKLVFFAEDVGSNKGAIIGLMVGGVVIA